(19) United States
(12) Patent Application Publication
(10) Pub. No.: US 2009/0220973 A1
(43) Pub. Date: Sep. 3, 2009

(54) OBESITY AND BODY FAT DISTRIBUTION

(75) Inventors: Stephane Gesta, Boston, MA (US); C. Ronald Kahn, West Newton, MA (US)

Correspondence Address: FISH & RICHARDSON PC, P.O. BOX 1022, MINNEAPOLIS, MN, 55440-1022, US

(73) Assignee: JOSLIN DIABETES CENTER, INC., Boston, MA (US)

(21) Appl. No.: 12/295,710

(22) PCT Filed: Apr. 3, 2007
(86) PCT No.: PCT/US2007/065853

§ 371 Date: Mar. 13, 2009

Related U.S. Application Data
(60) Provisional application No. 60/788,955, filed on Apr. 3, 2006.
(60) Provisional application No. 60/790,422, filed on Apr. 7, 2006.

(51) Int. Cl. C12Q 1/68

(52) U.S. Cl. 435/6

(57) ABSTRACT

Described are methods for predicting and diagnosing genetically-based obesity and body fat distribution, and for identifying compounds for the treatment and prevention of obesity.

### FEDERALLY SPONSORED RESEARCH OR DEVELOPMENT

This invention was made with Government support under Grant Nos. ROI DK33201, DK60837, and K08DK064906, awarded by the National Institutes of Health. The U.S. Government has certain rights in the invention.

### TECHNICAL FIELD

This invention relates to methods of predicting obesity and body fat distribution, and methods of identifying compounds for the treatment of obesity or the manipulation of body fat distribution.

### BACKGROUND

Obesity is an epidemic health problem worldwide that impacts on the risk and prognosis of many diseases, including diabetes, cardiovascular disease, hyperlipidemia, and cancer (Lean, (2000) Proc Nutr Soc 59, 331-6). However, not all obese patients have the same risk of developing these disorders. Individuals with peripheral obesity, i.e., fit distributed subcutaneously in the gluteofemoral region, are at little or no risk of the common medical complications of obesity, whereas individuals with central obesity, i.e., fat accumulated in visceral depots, are prone to these complications (Mauriege et al., (1993) Eur J Clin Invest 23, 729-40; Gillum, (1987) J Chronic Dis 40, 421-8; Kissebah and Krakower, (1994) Physiol Rev 74, 761-811; and Abate and Garg, (1995) Prog Lipid Res 34, 53-70).

While differentiation of adipocytes has been extensively characterized (Gregoire, (2001) Exp Biol Med (Maywood) 226, 997-1002; Koutnikova and Auwerx, (2001) Ann Med 33, 556-61; and Tong and Hotamisligil, (2001) Rev Endocr Metab Disord 2, 349-55) and there have been considerable recent insights into the control of appetite and energy expenditure as contributing factors to obesity (Wynne et al., (2005) J Endocrinol 184, 291-318; Ricquier, (2005) Proc Nutr Soc 64, 47-52), little is known about the genetic basis for determination of adipocyte number, differences in body fat distribution or their association with metabolic disorders. Twin and population studies have revealed that both body mass index (BMI) and waist-hip ratio (WHR) are heritable traits, with genetics accounting for 25-70% of the observed variability (Nelson et al., (2000) Twin Res 3, 43-50; and Baker et al., (2005) Diabetes 54, 2492-6). In addition, it is known that some obese individuals, especially those with early onset obesity, have increased numbers of adipocytes, but how these are distributed and why this occurs is unknown (Hirsch and Batchelor, (1976) Clin Endocrinol Metab 5, 299-311). Anecdotally, it is also clear that individual humans observe differences in their own body fat distribution as they gain or lose weight.

The uneven distribution of adipose tissue is extreme in some ethic groups, such as Hottentot women, who have been noted for excessive accumulation of fat in the buttocks, a condition known as steatopygia (Ersek et al., (1994) Aesthetic Plast Surg 18, 279-82). Striking differences in adipose tissue distribution can also be observed in individuals with partial lipodystrophy (Garg and Misra, (2004) Endocrinol Metab Clin North Am 33, 305-31), both in its acquired and inherited forms. For example, familial partial lipodystrophy of the Dunnigan type due to mutations in the Lamin A/C gene is characterized by a marked loss of subcutaneous adipose tissue in the extremities and trunk, without loss of visceral, neck or facial adipose tissue (Garg et al., (1999) J Clin Endocrinol Metab 84, 1704; Shackleton et al., (2000) Nat Genet 24, 153-6). Some lipodystrophies even appear to have a segmental or dermatomal distribution (Shelley and Izumi, (1970) Arch Dermatol 102, 326-9).

### SUMMARY

At least in pare the present invention is based on the discovery of major differences in expression of multiple genes involved in embryonic development and pattern specification between adipocytes taken from intra-abdominal and subcutaneous depots in rodents and humans. Similar differences were also present in the stromovascular fraction containing preadipocytes and that these differences persist in culture. Some of these developmental genes exhibit changes in expression that are closely correlated with level of obesity and the pattern of fat distribution.

In one aspect, the invention provides methods for diagnosing present obesity, e.g., high body mass index (BMI), or of predicting future obesity or undesirable adipose tissue distribution, e.g., high waist-hip ratio (WHR), in a subject, e.g., a human. The methods include providing a sample comprising a tissue or cell, e.g., an adipose tissue or cell, from the subject; and evaluating the level of mRNA in the cell for one, two, three, four or more of the genes listed in Table 1, e.g., one or more of Tbx15, Shox2, En1, Sfrp2, HoxC9, Nr2f1, Gpc4, Thbd, HoxA5 or HfoxC8, or a level of a protein encoded thereby. The level of expression, e.g., as compared to a predetermined reference level (e.g., as described herein), indicates whether the subject has, or is at risk of developing, obesity or undesirable adipose tissue distribution.

In some embodiments, the methods include determining a level of expression of at least one mRNA for a gene selected from the group consisting of Hox57, Gpc4 and Tbx15 in human adipose tissue, or a level of a protein encoded thereby, and comparing the levels to a reference, e.g., a reference that represents a subject with a selected BMI, e.g., a normal or near normal BMI. In some embodiments, the methods include measuring levels for one or both of Tbx15 in visceral fat and Gpc4 in subcutaneous fat.

In some embodiments, the relationship of the levels for the mRNA or protein in the human subject and the reference indicates whether the subject has or will develop an unhealthy BMI. The level of the mRNA or protein is used to select or exclude a subject for participation in a clinical trial.

In some embodiments, the subject is given a treatment or preventive measure for obesity, and the level of the mRNA or protein is correlated with the subject's response to the treatment or preventive measure for obesity. For example, the level of the protein or mRNA can be determined before, during and/or after the treatment, and a change in the level of the protein or mRNA indicates whether the subject is responding or has responded to the treatment.

In another aspect, the invention provides methods for determining a ratio of intra-abdominal (visceral) accumulation of fat versus subcutaneous (peripheral) fat in a subject. The methods include providing a first sample from the subject comprising visceral adipose cells or tissue; providing a second sample from the subject comprising peripheral adipose cells or tissue; quantifying a level of mRNA in the first and second samples for one, two, three, four or more of the genes listed in Table 1, e.g., one or more of Tbx15, Shox2, En1, Sfrp2, HoxC9, Nr2f1, Gpc4, Thbd, HoxA5 or HoxC8, or a level of a protein encoded thereby; and determining a ratio of the level of mRNA or protein in the first sample to the level of mRNA in the second sample. The ratio of the level of mRNA or protein in the first sample to the level of mRNA in the second sample indicates the ratio of visceral accumulation of fat versus peripheral fat in the subject. These methods can also be used to predict future undesirable distribution of weight.

In a her aspect, the invention provides methods for identifying a candidate compound, e.g., for the treatment of obesity. The methods include providing a sample comprising an adipose cell or tissue expressing one, two, three, four or more of the genes listed in Table 1, e.g., one or more of Tbx15, Shox2, En1, Sfrp2, HoxC9, Nr2f1, Gpc4, Thbd, HoxA5 or HoxC8; contacting the cell or tissue with a test compound, e.g., a small organic or inorganic molecule, an inhibitory or stimulatory nucleic acid, or a polypeptide; and evaluating the expressing of the one, two, three, four or more of the genes listed in Table 1, e.g., one or more of Tbx15, Shox2, En1, Sfrp2, HoxC9, Nr2f1, Gpc4, Thbd, HoxA5 or HoxC8, in the cell. A test compound that appropriately-modulates the expression of the gene or genes is a candidate compound for the treatment of obesity.

Further, the invention provides additional methods for identifying a candidate compound, e.g., for the treatment of obesity. The methods include providing a sample comprising one, two, three, four or more proteins expressed by a gene listed in Table 1, e.g., one or more of Tbx15, Shox2, En1, Sfrp2, HoxC9, Nr2f1, Gpc4, Thbd, HoxA5 or HoxC8, or a cell or tissue expressing the proteins; contacting the sample with a test compound, e.g., a small organic or inorganic molecule, an inhibitory or stimulatory nucleic acid, or a polypeptide; and evaluating the level or activity of the protein in the sample. A test compound that appropriately modulates, e.g., increases or decreases, the level or activity of the protein is a candidate compound for the treatment of obesity.

Unless otherwise defined, all technical and scientific terms used herein have the same meaning as commonly understood by one of ordinary skill in the art to which this invention belongs. Exemplary methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a schematic illustration of the experimental design used in some of the examples set forth herein. Flank subcutaneous and intra-abdominal (epididymal) white adipose tissues were taken from 6-7 week old pooled C5Tb1/6 males. Stromovascular fraction and adipocytes were isolated after collagenase digestion of adipose tissues. Equal quantities of RNA were isolated from isolated adipocytes and stromovascular fraction of each fat depot. A hybridization mixture containing 15 μg of biotinylated cRNA, adjusted for possible carryover of residual total RNA, was prepared and hybridized to mouse Affymetrix U74Av2 chips.

FIG. 1B is a diagram illustrating some of the results described herein. Among the 12,488 probesets present on the U74Av2 chip, 8,017 probesets representing 6174 are annotated for Gene Ontology Biological Process. Significant genes with differential expression in both depots were identified by selecting genes that passed two independent filters of significance (p-value Student's t-test <0.05 and pFDR <0.05) (see Methods). The first filter p-value Student's t-test <0.05) selected 1,276 genes differentially expressed in the stromovascular fraction, 537 genes differentially expressed in isolated adipocytes and 233 genes differentially expressed in both cell fractions. Of these 233 genes, 197 genes passed the second filter of significance (PFDR <0.05) and were assessed against an a priori set of 198 annotated genes involved in embryonic development and pattern specification (see Methods). Twelve genes from this set were found among the differentially expressed genes.

FIGS. 2A-C are bar graphs illustrating the results of a comparison of Tbx15, Shox2, En1, Sfrp2 and HoxC9 gene expression between intra-abdominal (Epi; opened bars) and subcutaneous (SC; closed bars) adipose tissue of C57316 mice performed using real time PCR. These genes had a higher level of expression in subcutaneous in whole adipose tissue (2A) (Epi versus Sc; * p-value <0.05), isolated adipocytes and stromovascular fraction (2B) (Epi versus Sc; * p-value <0.05). These differences of expression were maintained when stromovascular fraction taken from intra-abdominal (epididymal) or subcutaneous adipose were placed in culture in a defined serum free medium and subjected to in vitro differentiation (2C) suggesting these differences are independent of extrinsic factors (Epi versus Sc; * p-value <0.05)

FIGS. 3A-3C are bar graphs illustrating the results of a comparison of Nr2f1, Gpc4, Thbd, HoxA5 and HoxC8 gene expression between intra-abdominal (Epi; opened bars) and subcutaneous (SC; closed bars) adipose tissue of C57B16 mice performed using real time PCR. These genes had a higher level of expression in intra-abdominal (epidydimal) whole adipose tissue (3A) (Epi versus Sc; * p-value <0.05), isolated adipocytes and stromovascular fraction (3B) (Bpi versus Sc; * p-value <0.05). These differences of expression were maintained when stromovascular fraction taken from intra-abdominal (epididymal) or subcutaneous adipose were placed in culture in a defined serum free medium and subjected to in vitro differentiation (3C) suggesting these differences are independent of extrinsic factors (Epi versus Sc; * p-value <0.05)

FIGS. 4A-4J are bar graphs illustrating differential expression of subcutaneous dominant genes and intra-abdominal dominant genes in subcutaneous and intra-abdominal adipose tissue of lean humans. Visceral (Vis, opened bars) and subcutaneous (SC, closed bars) adipose tissue biopsies were performed on 53 lean subjects (BMI<25; 22 males and 31 females). Shox2 (4A), En1 (4B), HoxC9 (4C), Sfrp2 (4D), Tbx15 (4E), Nr2f1 (4F), Thbd (4G), HoxA5 (4H), HoxC8 (4I), and Gpc4 (4J) expression levels were compared in both depots using real time PCR (Vis versus SC, * p<0.05).

FIGS. 5A and 5B are each sets of six scatter/line graphs illustrating expression of HoxA5, Gpc4 and Tbx15 in subcutaneous and visceral adipose tissue in human are correlated with adiposity and fat distribution. One hundred ninety eight subjects (99 males and 99 females) ranging from lean to obese with variable BMI (5A) and fat distribution (WHR) (5B) were subjected to visceral (Vis, opened bars) and subcutaneous (SC, closed bars) adipose tissue biopsies. Gene expression of HoxA5 (top panels), Gpc4 (middle panels) and Tbx15 (bottom panels) was assessed in both fat depots by real time PCR as described in Materials and Methods. Correlation significances were determined using Stat View software, either as linear correlations or in the case of non-linear correlations by exponential or lowess curve fitting.

FIG. 6 is a schematic diagram illustrating a hypothetical scheme of adipocyte development, not meant to be limiting.

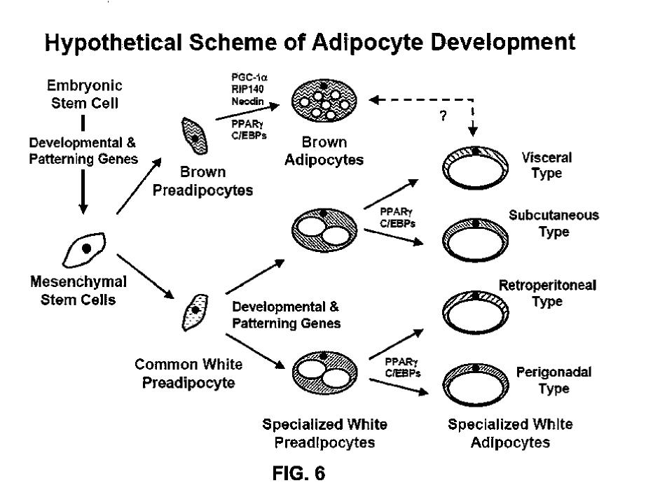

### DETAILED DESCRIPTION

Obesity is a multifactorial disorder influenced by a mixture of genetic and environmental factors, including control of appetite and energy expenditure, availability and nutritional content of food, and development of adipocyte cell mass. Furthermore, obesity occurs with different degrees of fat accumulation in different depots, and these are associated with different metabolic consequences with intra-abdominal (visceral) accumulation of fat producing a much greater risk of diabetes, dyslipidemia and accelerated atherosclerosis than subcutaneous (peripheral) fat. The accumulation of visceral fat, e.g., as opposed to peripheral fat, is referred to herein as “undesirable body fat distribution.”

Although obesity and body fat distribution are clearly hereditable traits, the role of developmental genes in obesity and fat distribution has received surprisingly little attention. Stromovascular fractions taken from different adipose depots (Djian et al., (1983) J Clin Invest 72, 1200-8, Adams et al., (1997) J Clin Invest 100, 3149-53; Kirkland et al., (1990) Am J Physiol 258, C206-10; Hauner and Entenmann, (1991) Int J Obes 15, 121-6; Tchkonia et al., (2002) Am J Physiol Regul Integr Comp Physiol 282, R1286-96; and Tchkonia et al., (2005) Am J Physiol Endocrinol Metab 288, E267-77) and from obese versus lean individuals show differing propensity to differentiate when place in tissue culture in vitro (van Harmelen et al., (2003) Int J Obes Relat Metab Disord 27, 889-95). In addition, the rate of lipolysis in adipose tissue taken from subcutaneous sites is lower than of adipose tissue from visceral or omental sites (Amer, (1995) Ann Med 27, 435-8). Furthermore, the lipolytic effect of catecholamines is weaker and the antilipolytic effect of insulin is more pronounced in subcutaneous as compared to visceral adipose tissue (Mauriege et al., (1987) Fur J Clin Invest 17, 156-65; and Bolinder et al., (1983) Diabetes 32, 117-23).

Characterization of differences in gene expression between human subcutaneous and visceral adipose tissue also suggest genetic/developmental heterogeneity. Acylation stimulating protein and angiotensinogen mRNA levels are higher in visceral adipose, whereas the levels of leptin, PPARγ, GLUT4, glycogen synthase and cholesterol ester transfer protein (CETP) are higher in the subcutaneous depot (Lefebvre et al., (1998) Diabetes 47, 98-103; and Dusserre et al., (2000) Biochim Biophys Acta 1500, 88-96). In a survey of genes differentially expressed in subcutaneous and visceral adipose tissue in men, Vohl et al. ((2004) Obes Res 12, 1217-22) also noted differences in genes involved in lipolysis, cytokine secretion, Wnt signaling, C/EPBα and some HOX genes. Differences in large and small adipocytes taken from normal and fat insulin receptor knockout (FIRKO) mice with regard to function, gene and protein expression have also been observed (Bluher et al., (2002) Dev Cell 3, 25-38; Bluher et al., (2004) J Biol Chem 279, 31891-901; and Bluher et al., (2004) J Biol Chem 279, 31902-9). The present study, therefore, explored the hypothesis that developmental genes might play an important role in obesity and body fat distribution in both rodents and humans.

Using microarray and qPCR analysis, 197 genes were found to be differentially expressed in both adipocytes and SVF-containing preadipocytes from subcutaneous and intra-abdominal depots of the mouse; of these, at least 12 are genes previously thought to play a role in early development and pattern specification. Of these, Tbx15, Shox2, En1, Sfrp2 and HoxC9 were more highly expressed in cells of subcutaneous adipose tissue, whereas Nr2f1, Gpc4, Thbd, HoxA5 and HoxC8 were more expressed in intra-abdominal adipose tissue. These differences in gene expression are intrinsic and persist during in vitro culture and differentiation indicating that they are cell autonomous and independent of tissue microenvironment. Since the expression of these developmental genes emerges during embryogenesis, before any white adipose tissue can be detected, and is maintained during adult life, this suggests that different adipocyte precursors are responsible for a specific adipose depot development and may participate later in the functional differences observed between internal and subcutaneous adipose depots.

Methods of Diagnosis

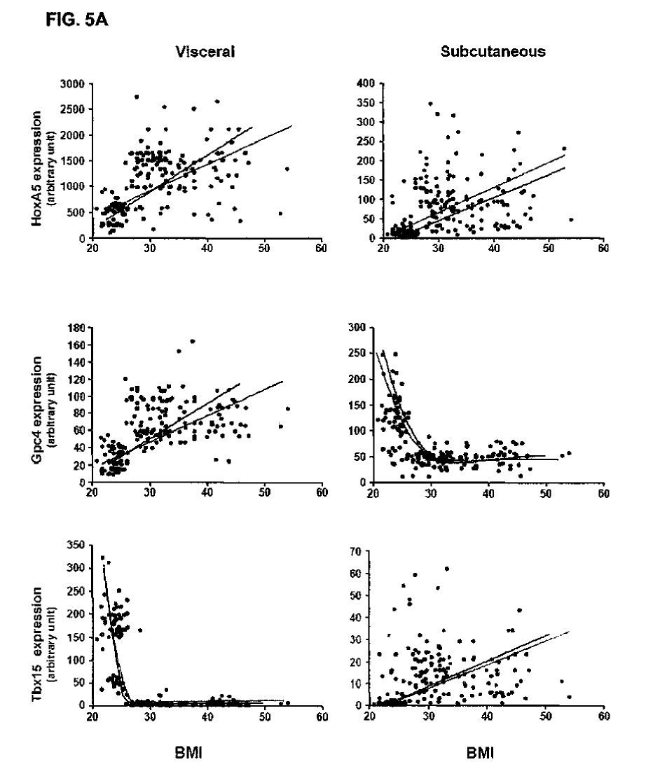

Included herein are methods for diagnosing obesity, for quantifying distribution of body fat, and for predicting fixture obesity and undesirable body fat distribution. The methods include obtaining a sample from a subject, e.g., a sample comprising a brown or white adipocyte or preadipocyte, and evaluating the presence and/or level of one, two, three, four or more of the genes listed in Table 1, e.g., one or more of Tbx15, Shox2, En1, Sfrp2, HoxC9, Nr2f1, Gpc4, Thbd, HoxA5 or HoxC8 in the sample, and comparing the presence and/or level with one or more references, e.g., a control reference that represents a normal level of the gene or genes, e.g., a level in an unaffected subject, and/or a disease reference that represents a level of the proteins associated with obesity or undesirable distribution of body fat, e.g., a level in a subject having excessive amounts of visceral fat. Suitable reference values can include those shown in FIG. 4/Example 4.

Differential Gene Expression in Mice and Humans

While all of the genes that were differentially expressed in rodents were also differentially expressed in humans, in some cases, the direction of difference was different in the two species. This may reflect the fact that fat was not taken from identical depots in the two species or may simply represent differences between development in these two species. Other differences in gene expression have also been observed between humans and rodents. Thus, leptin exhibits a higher expression in subcutaneous than omental adipose in humans (Lefebvre et al., (1998) Diabetes 47, 98-103; and Dusserre et al., (2000) Biochim Biophys Acta 1500, 88-96), whereas in mice, leptin expression is higher in intra-abdominal (epididymal) fat than subcutaneous fat (Trayhurn et al., (1995) FEBS Let 368, 488-90). Likewise, the differential expression of α₂-adrenergic receptor expression observed in humans (higher in subcutaneous adipose than in omental) Mauriege et al., (1987) Eur J Clin Invest 17, 156-65) is not observed at all in mice, which do not express α₂-adrenergic receptors in adipose tissue (Castan et al., (1994) Am J Physiol 266, R1141-7). Conversely, β₃-adrenergic receptors are widely expressed in mouse adipose tissue, whereas little or no expression has been reported in human adipose (Lafontan (1994) Cell Signal 6, 363-92). In our case, the interdepot differences of expression for developmental genes Shox2, En1, Nr2f1, HoxA5. HoxC8 and Thbd were preserved from mice to humans independent of gender, whereas interdepot differential expression of HoxC9 in humans occurred only in females, and Tbx15, Sfrp2 and Gpc4 exhibited opposite directions of differential expression in mice and humans. In both species, what is clear is that multiple developmental genes, including those involved in antero-posterior or dorso-ventral patterning, exhibit dramatic differences in level of expression in adipose and preadipose from different regions of the body.

Correlation of Gene Expression with Body Mass Index (BMI)

One of the most striking features of the expression of HoxA5, Gpc4 and Tbx15 in human adipose is not only their differential expression between depots, but their strong correlation with BMI. This is particularly true for Tbx15 in visceral fat and Gpc4 in subcutaneous fat such that both genes show dramatic changes in expression as BMI goes from the normal range (20-25) to either overweight (25-30) or obese (>30).

No other parameter related to obesity or fat mass, including serum leptin, adiponectin or insulin, shows such a distinct change at this transition point. Indeed, if the physiological separation between lean and overweight/obese had not been previously defined by epidemiological criteria, one could define the overweight population by the expression level of these genes, suggesting that expression of these genes could related to the pathogenesis of obesity.

Thus, the methods described herein include determining levels of HoxA5; Gpc4 and Tbx15 in human adipose tissue, and comparing the levels to a reference, e.g., a reference that represents a subject with a selected BMI, e.g., a normal or near normal BMI. In some embodiments, the methods include measuring Tbx15 in visceral fat and/or Gpc4 in subcutaneous fat. The relationship of the levels of the genes in the human subject and the reference can be used to diagnose present obesity or predict the future likelihood that the subject will develop an unhealthy BMI. The levels of these genes can also be used to select subjects, e.g., stratify subjects, for participation in a clinical trial, and to correlate their expression with response to a given treatment or preventive measure for obesity.

Correlation of Gene Expression with Waist-Hip Ratio (WHR)

Distribution of adipose tissue (WHR) also has a strong heritable component (Baker et al., (2005) Diabetes 54, 2492-6) and has been shown to better correlate with risk of diabetes and atherosclerosis than BMI (Ohlson et al., (1985) Diabetes 34, 1055-8). Increased WHR, i.e., visceral/central or “apple shaped” obesity, also referred to herein as undesirable body fat distribution, is associated with higher risks for metabolic and cardiovascular complications (Mauriege et al., (1993) Eur J Clin Invest 23, 72940; Gillum, (1987) J Chronic Dis 40; 421-8; Kissebah and Krakower, (1994) Physiol Rev 74, 761-811; Abate and Garg, (1995) Prog Lipid Res 34, 53-70). Ideally, women should have a waist-to-hip ratio of 0.8 or less, and men should have a waist-to-hip ratio of 0.95 or less.

As described herein, HoxA5, Gpc4 and Tbx15 expression also vary with fat distribution, and that expression of the latter two is an excellent marker for visceral fat accumulation. Thus, high levels of Tbx15 and Gpc4 expression in subcutaneous adipose tissue and low levels of expression in visceral adipose tissue appear to be linked with high WHR and by extension should be correlated with higher risks for metabolic and cardiovascular complications.

Therefore, the methods described herein include evaluating the expression levels of these genes in adipose cells taken from different sites in the body, e.g., subcutaneous versus visceral fat depots, and comparing the expression levels to a reference level, a reference level that represents a subject with normal or close to normal body fat depots in the corresponding sites in the body. The difference between the level of expression of the gene in the subject's cells versus the reference will indicate whether there is, or in the future will be, an excessive (or insufficient) amount of adipose tissue in the relevant part of the body. As one example, levels of a gene that is listed in Table 1 for which increased expression is associated with increased adipose tissue will be indicative of increased adipose deposits if the level in the subject are above those in the reference. The converse is true for those genes for which decreased expression is associated with increased adipose depots. Thus, the methods described herein include determining levels of HoxA5, Gpc4, and Tbx15 in human adipose tissue, and comparing the levels to a reference, e.g., a reference that represents a subject with a selected WHR, e.g., a normal or near normal WHR. The relationship of the levels of the genes in the human subject and the reference can be used to diagnose present obesity or predict the future likelihood that the subject will develop an unhealthy WHR. The levels of these genes can also be used to select subjects, e.g., stratify subjects, for participation in a clinical trial, and to correlate their expression with response to a given treatment or preventive measure for obesity.

The methods can also include using standard mathematical algorithms to determine the ratio of expression of a given gene in the different fat depots, e.g., a ratio of expression between subcutaneous and visceral tissues, and comparing that ratio to a reference ratio, e.g., reference ration that represents a subject with normal or close to normal body fit distribution. Again, depending on whether increased or decreased expression of the gene is associated with increased adipose tissue depots, the relationship between the ratio in the subject and the reference ratio will be indicative of the presence or future likelihood of developing undesirable body fat distribution. The levels of these genes can also be used to select subjects, e.g., stratify subjects, for participation in a clinical trial, and to correlate their expression with response to a given treatment or preventive measure for obesity or undesirable body fat distribution.

For example, the methods can include measuring HoxA5, Gpc4 and Tbx15 in visceral fat and in subcutaneous fat. High levels of Tbx15 and Gpc4 expression in subcutaneous adipose tissue and low levels of expression in visceral adipose tissue indicate the presence or future likelihood of high WHR, and therefore higher risk for metabolic and cardiovascular complications

In some embodiments, the presence and/or level of the one or more genes is comparable to the presence and/or level of the one or more genes in the disease reference, and the subject has one or more symptoms or risk factors associated with obesity or undesirable body fat distribution, then the subject has, or is at an increased risk for, obesity or undesirable body fat distribution. In some embodiments, the subject has no overt signs or symptoms of obesity or undesirable body fat distribution, but the presence and/or level of one or more of the proteins evaluated is comparable to the presence and/or level of the protein(s) in the disease reference, then the subject has an increased risk of developing obesity or undesirable body fat distribution. For example, in a subject who is adolescent or pre-adolescent, the presence of a pathological level of the one or more genes may indicate that the subject is at an increased risk of future obesity or undesirable body fat distribution.

In some embodiments, the sample includes an adipose cell. In some embodiments, once it has been determined that a person has obesity or undesirable body fat distribution, or has an increased risk of developing obesity or undesirable body fat distribution, then a treatment, e.g., as known in the art or as described herein, can be administered.

Assay Methods

The presence and/or level of a gene or protein can be evaluated using methods known in the art, e.g., using standard Northern or Western analysis. In some embodiments, high throughput methods, e.g., protein or gene chips as are known in the art (see, e.g., Ch-12, “Genomies,” in Griftis et al., Eds. Modern genetic Analysis, 1999, W.H. Freeman and Company; Ekis and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of the one or more genes.

In addition, methods for detecting or evaluating the activity of a selected protein are known in the art, and will vary depending on the protein selected.

Methods of Screening

The invention includes methods for screening of test compounds, to identify compounds that modulate the expression of one, two, three, four or more of the genes listed in Table 1, e.g., one or more of Tbx15, Shox2, En1, Sfrp2, HoxC9, Nr2f1, Gpc4, Thbd, HoxA5 or HoxC8, in a cell, e.g., an adipose cell, e.g., a brown or white adipocyte or preadipocyte. Assay methods useful in the methods of screening are described herein and known in the art.

In some embodiments, the test compounds are initially members of a library, e.g., an inorganic or organic chemical library, peptide library, oligonucleotide library, or mixed-molecule library. In some embodiments, the methods include screening small molecules, e.g., natural products or members of a combinatorial chemistry library.

A given library can comprise a set of structurally related or unrelated test compounds. Preferably, a set of diverse molecules should be used to cover a variety of frictions such as charge, aromaticity, hydrogen bonding, flexibility, size, length of side chain, hydrophobicity, and rigidity. Combinatorial techniques suitable for creating libraries are known in the a, e.g., methods for synthesizing libraries of small molecules, e.g., as exemplified by Obrecht and Villalgordo, Solid-Supported Combinatorial and Parallel Synthesis of Small-Molecular-Weight Compound Libraries, Pergamon-Elsevier Science Limited (1998). Such methods include the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of libraries, including small molecule libraries, are commercially available.

In some embodiments, the test compounds are peptide or peptidomimetic molecules, e.g., peptide analogs including peptides comprising non-naturally occurring amino acids or having non-peptide linkages; peptidomimetics (e.g., peptoid oligomers, e.g., peptoid amide or ester analogues, β-peptides, D-peptides, L-peptides, oligourea or oligocarbamate); small peptides (e.g., pentapeptides, hexapeptides, heptapeptides, octapeptides, nonapeptides, decapeptides, or larger, e.g., 20-mers or more); cyclic peptides; other non-natural or unnatural peptide-like structures, and inorganic molecules (e.g., heterocyclic ring molecules). In some embodiments, the test compounds are nucleic acids, e.g., DNA or RNA oligonucleotides.

In some embodiments, test compounds and libraries thereof can be obtained by systematically altering the structure of a first test compound. Taking a small molecule as an example, e.g., a first small molecule is selected that has been identified as capable of modulating the expression of one, two, three, four or more of the genes listed in Table 1, e.g., one or more of Tbx15, Shox2, En1, Sfrp2, HoxC9, Nr2f1, Gpc4, Thbd, HoxA5 or HoxC8. For example, in one embodiment, a general library of small molecules is screened, e.g., using the methods described herein, to select a fist test small molecule. Using methods known in the art, the structure of that small molecule is identified if necessary and correlated to a resulting biological activity, e.g., by a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds.

In some embodiments, test compounds identified as “hits” (e.g., test compounds that demonstrate the ability to modulate one, two, three, four or more of the genes listed in Table 1, e.g., one or more of Tbx15, Shox2, En1, Sfrp2, HoxC9, Nr2f1, Gpc4, Thbd, HoxA5 or HoxC8) in a first screen are selected and optimized by being systematically altered, e.g., using rational design, to optimize binding affinity, avidity, specificity, or other parameter. Such potentially optimized structures can also be screened using the methods described herein. Thus, in one embodiment the invention includes screening a first library of test compounds using a method described herein, identifying one or more hits in that library, subjecting those hits to systematic structural alteration to create one or more second generation compounds structurally related to the hit, and screening the second generation compound. Additional rounds of optimization can be used to identify a test compound with a desirable therapeutic profile.

Test compounds identified as hits can be considered candidate therapeutic compounds, useful in treating disorders described herein. Thus, the invention also includes compounds identified as “hits” by a method described herein, and methods for their administration and use in the treatment, prevention, or delay of development or progression of a disease described herein.

### EXAMPLES

The invention is further described in the following examples, which do not limit the scope of the invention described in the claims.

### Example 1

### Genes Expression Differences Between Intra-Abdominal And Subcutaneous Adipose Tissue of Mice

Several studies have reported differences in gene expression (Atzmon et al, (2002) Horm Metab Res 34, 622-8; Linder et al., (2004) J Lipid Res 45, 148-54; Vohl et al., (2004) Obes Res 12, 1217-22; and von Eyben et al., (2004) Ann N Y Acad Sci 1030, 508-36) and proliferative capacity (Djian et al., (1983) J Clin Invest 72, 1200-8; Adams et al., (1997) J Clin Invest 100, 3149-53; Kirkland et al., (1990) Am J Physiol 258, C206-10; Hauner and Entenmann, (1991) Int J Obes 15, 121-6; Tchkonia et al., (2002) Am J Physiol Regul Integr Comp Physiol 282, RI 286-96; and Tchkonia et al., (2005) Am J Physiol Endocrinol Metab 288, E267-77) between fat taken from different depots in rodents and humans suggesting that genetic pro n g could affect specific adipose depot development.

To address this hypothesis, we performed gene expression analysis of both adipocytes (Ad) and stromovascular fraction (SVF) containing preadipocytes taken from subcutaneous (flank) fat and intra-abdominal (epididymal) fat.

Embryonic Development and Pattern Specification Set of Genes

A priori, we created a set of genes involve in embryonic development and pattern specification, using Gene Ontology Biological Processes annotations. The NetAffx™ Analysis Center (available on the world wide web at affymetrix.com), was queried for genes annotated for “embryonic development,” “pattern specification,” “pattern formation,” “mesoderm formation,” and/or “organogenesis.” The list obtained was then scrutinized and updated by review of the relevant literature for data implicating each gene family in directing embryonic development (e.g., BMP family, Frizzled homolog family, Hox family, or Pax family). A final set of 198 genes (254 probesets) with strong literature support was thus chosen to evaluate the enrichment in genes involve in embryonic development and pattern specification.

Adipose Tissue, Isolated Adipocytes and Stromovascular Fractions (SVF) and RNA Extraction

For analysis of adipose tissue, Six 6 to 7 weeks old C57b1/6 males were sacrificed and epididymal and flank subcutaneous adipose tissue were removed, washed with PBS, and immediately subjected to RNA extraction. To obtain purified cell fractions, ten 6- to 7-weeks old C57b1/6 males were sacrificed and epididymal and flank subcutaneous adipose tissue were removed under sterile conditions. Tissues from each depot were pooled, minced and digested with 1 mg/ml collagenase for 45 minutes at 37° C. in Dulbecco's modified Eagle's medium/Hamn's F-12 1:1 (DMEM/F 12), containing 1% BSA and antibiotics (penicillin 100 U/ml, streptomycin 0.1 mg/ml, fungizone 2.5 μg/ml and gentamicin 50 μg/ml). Digested tissues were filtered through sterile 150 μm nylon mesh and centrifuged at 250×g for 5 minutes. The floating fraction consisting of pure isolated adipocytes was then removed and washes 2 more times before proceeding to RNA extraction. The pellet, representing the stromovascular fraction containing preadipocytes and other cell types, was resuspended in erythrocyte lysis buffer consisting of 154 mM NH₄Cl, 10 mM KHCO₃ and 0.1 mM EDTA for 10 minutes. The cell suspension was centrifuged at 500×g for 5 minutes and then resuspended in a culture medium consisting DMEM/F12, 10% fetal calf serum (FCS) and antibiotics. This cell suspension was filtered through a 25 μm sterile nylon mesh before being plated on 10 cm plate at 60,000 cells per cm². 16 hours after plating, cells were extensively washed with PBS then subjected to RNA extraction.

Sample Preparation for Microarrays

RNA from adipose tissue, isolated adipocytes and stromovascular fractions were isolated using RNeasy kit (Qiagen). Double-stranded cDNA synthesis was reverse transcribed from 15 μg of isolated mRNA using the SuperScript Choice system (Invitrogen) using an oligo(dT) primer containing a T7 RNA polymerase promoter site. Double-stranded cDNA was purified with Phase Lock Gel (Eppendorf). Biotin-labeled cRNA was transcribed using a BioArray™ RNA transcript labeling kit (Enzo). A hybridization mixture containing 15 μg of biotinylated cRNA, adjusted for possible carryover of residual total RNA, was prepared and hybridized to mouse Affymetrix MG-U74A-v2 chips. The chips were washed, scanned, and analyzed with GeneChip® MAS Microarray Suite Software V. 5.0. For each group (epididymal and subcutaneous), 3 chips, each representing a pool of RNA from 10 mice, was analyzed used. All chips were subjected to global scaling to a target intensity of 1500 to take into account the inherent differences between the chips and their hybridization efficiencies.

Microarray Analysis

The 8017 probesets on the murine U74Av2 microarray representing 6174 genes with annotations for Gene Ontology biologic processes (available on the internet at affymetrix.com, accessed Nov. 13, 2005) were considered for analysis. To obtain a list of genes with a conjoint differential expression between the two tissue beds, we selected genes that passed two independent filters of significance. The first filter screened for genes with evidence of independent differential expression for both tissues types between tissue beds by selecting those genes with significance levels of p<0.05 using Student's t-test for both cell types (Ae versus Ase; Se versus Ssc). The second filter used a single test statistic to selected genes that exhibited concordant and significant differential expression in both the adipocytes and stromovascular fractions between epididymal and subcutaneous adipose tissues. To this end, we used a combined test statistic T_{Δ+Δ}=[(S_{sc}−Sₑ)+(A_{sc}−Aₑ)]/SD, where S represents the expression value in the stromovascular fraction, A adipocytes, subscript sc subcutaneous depot subscript e epididymal depot, and SD the sums of the four standard deviations. T_{Δ+Δ} is expected to be zero when there is no difference in expression between tissue depots, and non-zero if one cell-type experienced differential expression between tissue depots. Congruent changes in expression between tissue depots for both cell types will lead to even greater values of T_{Δ+Δ}, whereas changes of opposite direction will cancel each other out. By using this single test statistic, we were able to determine the positive false discovery rate (pFDR) (Mauriege et al., (1993) Eur J Clin Invest 23, 729-40), thus determining the probability of significant joint differential regulation corrected for multiple hypothesis testing.

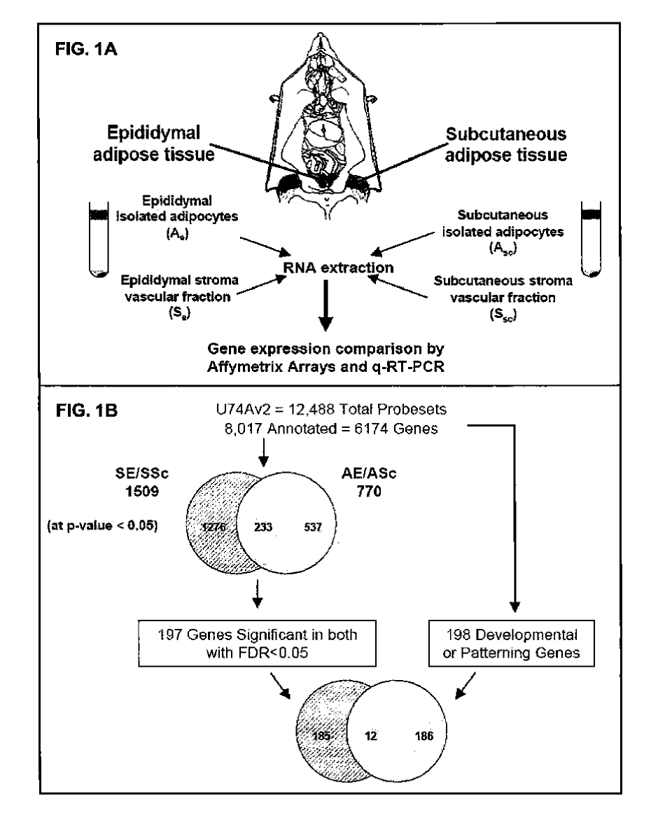

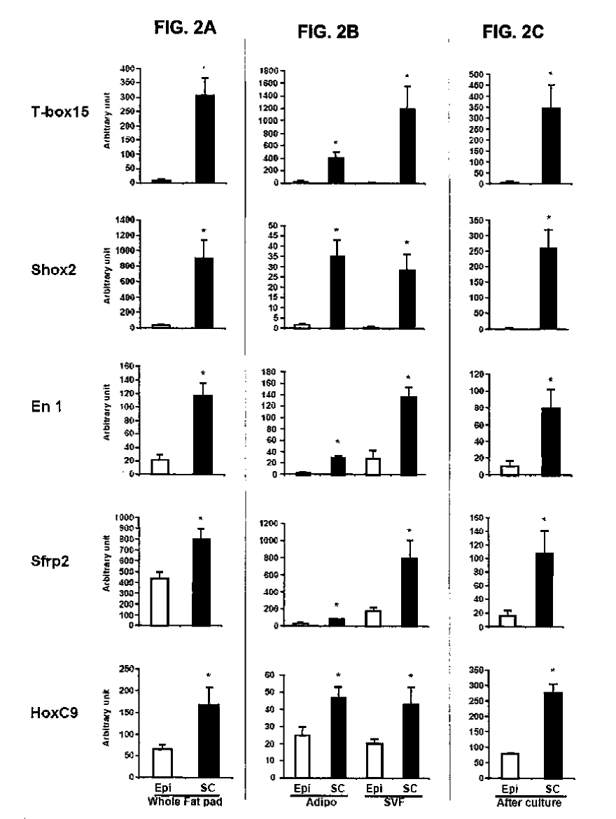

The Affymetrix U74Av2 microarrays were used, with 8017 probesets representing 6174 different annotated genes (www.affymetrix.com, Nov. 13, 2005) (FIG. 1B). Of these, 197 genes were found to have conjoint differential expression in both cell fractions between the two tissue beds using stringent statistical criteria with a two tailed t-test value for both cell fractions <0.05 and a positive False Discovery Rate value <0.05 (see Table 1). This list was assessed against an a priori set of 198 annotated genes involved in embryonic development and pattern specification on the array (see Table 2). Twelve of these developmental genes were found among the differentially expressed genes, representing a 1.9-fold enrichment (=0.006) compared to the 6174 annotated genes on the array (Table 1).

Among these 12 genes, seven genes had higher levels of expression in intra-abdominal a epididymal SVF and/or adipocytes (Nr2f1, Thbd, HoxA5, HoxC8, Gpc4, Hrmt112, and Vdr) and five genes had higher levels of expression in subcutaneous SVF and/or adipocytes (Tbx15, Shox2, En1, Slpr2 and HoxC9). Of the seven genes from intra-abdominal group, we decided to focus our analysis on the five most significant genes, including two Homeobox genes, HoxA5 and HoxC8; Nr2f1, nuclear receptor subfamily 2 group F member 1, also known as COUP-TFI, an orphan member of the steroid receptor superfamily thought to be involved in organogenesis (Pereira et al., (199S) J Steroid Biochem Mol Biol 53, 503-8); glypican 4 (Gpc4), a cell surface heparan sulfate proteoglycan involved in cell division and growth regulation (De Cat and David, (2001) Semin Cell Dev Biol 12, 117-25); and thrombomodulin (Thbd), a surface glycoprotein of endothelial and placental cells (Weiler and Isermann, (2003) J Thromb Haemost 1, 1515-24). All five genes from subcutaneous group of genes including the Homeobox gene HoxC9; short stature Homeobox 2 (Shox2) a transcription factor with homeodomain expressed during embryonic development (Blaschke et al., (1998) Proc Natl Acad Sci USA 95, 2406-11); Thox-15 (Tbx15), a transcription factor involved in craniofacial and limb development in the mouse (Singh et al., (2005) Mech Dev 122, 131-44); engrailed 1 (En1), the mouse homologue of a Drosophila patterning gene (Joyner and Martin, (1987) Genes Dev 1, 29-38); and secreted frizzled-related protein 2 (Sfrp2), a soluble modulator of Wnt signaling (Leimeister et al., (1998) Mech Dev 75, 29-42), were also studied.

TABLE 1
Genes Showing Differential Expression in Adipoctyes
and Stromovascular Fraction of Adipose Tissue.

| | | Mean | Mean | p value | Mean | Mean | p value |
|---|---|---|---|---|---|---|---|
| Gene ID | Probeset ID | Ae | Asc | Ae vs Asc | Se | Ssc | Se vs Ssc |
| AE000663 | 92712_at | 99.3 | 389.9 | 0.0171 | 241.8 | 359.6 | 0.0039 |
| AA675604 | 104544_at | 162.4 | 773.7 | 0.0469 | 129.7 | 838.2 | 0.0012 |
| AI849271 | 95064_at | 50538.9 | 77559.2 | 0.0285 | 7387.2 | 4569.3 | 0.0123 |
| J04946 | 160927_at | 219.1 | 662.0 | 0.0364 | 314.5 | 2798.3 | 0.0020 |
| AI838021 | 97456_at | 1398.1 | 2474.9 | 0.0201 | 1723.4 | 2838.2 | 0.0039 |
| AF045887 | 101887_at | 16339.4 | 2185.5 | 0.0078 | 88.7 | 754.0 | 0.0021 |
| AB027125 | 95015_at | 2059.1 | 790.5 | 0.0047 | 652.9 | 148.7 | 0.0149 |
| M74570 | 100068_at | 17238.1 | 8658.5 | 0.0063 | 25048.8 | 12039.6 | 0.0076 |
| AW123269 | 96784_at | 383.4 | 72.0 | 0.0016 | 3239.2 | 640.8 | 0.0096 |
| AV003419 | 161703_f_at | 44613.5 | 26734.9 | 0.0272 | 33384.2 | 12585.7 | 0.0010 |
| M14044 | 100569_at | 98848.5 | 53546.4 | 0.0099 | 30093.7 | 18292.6 | 0.0384 |
| AJ002390 | 97529_at | 1219.7 | 3507.0 | 0.0336 | 477.5 | 1645.1 | 0.0123 |
| M97216 | 93498_s_at | 24056.5 | 18497.4 | 0.0046 | 7375.0 | 4106.1 | 0.0248 |
| X82648 | 93592_at | 87.8 | 335.6 | 0.0094 | 95.2 | 274.4 | 0.0287 |
| D87901 | 160082_s_at | 9205.1 | 5438.4 | 0.0211 | 18123.6 | 12870.6 | 0.0026 |
| AI852332 | 99497_at | 804.8 | 1164.0 | 0.0319 | 583.5 | 1371.2 | 0.0103 |
| AI846773 | 104315_at | 2814.7 | 1389.7 | 0.0376 | 6232.6 | 1996.7 | 0.0007 |
| M63725 | 100984_at | 1498.0 | 1162.9 | 0.0153 | 3211.6 | 5035.8 | 0.0367 |
| U13840 | 92603_at | 7219.7 | 5807.5 | 0.0271 | 7956.1 | 4528.0 | 0.0015 |
| X01838 | 93088_at | 68009.1 | 78627.3 | 0.0219 | 40904.2 | 64767.4 | 0.0072 |
| X55573 | 102727_at | 119.3 | 370.4 | 0.0390 | 8004.2 | 2198.5 | 0.0000 |
| D83745 | 96146_at | 874.5 | 1375.1 | 0.0064 | 7989.1 | 3765.1 | 0.0165 |
| X06454 | 103033_at | 18499.6 | 33207.4 | 0.0016 | 4763.7 | 42308.8 | 0.0000 |
| M19381 | 96522_at | 40446.4 | 28714.6 | 0.0090 | 20940.0 | 12234.6 | 0.0174 |
| M27844 | 93293_at | 27546.3 | 20459.3 | 0.0176 | 49178.8 | 40506.3 | 0.0259 |
| AI842328 | 92632_at | 4473.1 | 3046.9 | 0.0244 | 8109.3 | 4285.6 | 0.0121 |
| U16740 | 93499_at | 5064.7 | 4173.5 | 0.0313 | 9309.7 | 8050.9 | 0.0080 |
| U16741 | 98127_at | 5755.6 | 3678.1 | 0.0107 | 5109.3 | 3526.1 | 0.0067 |
| AI747654 | 160280_at | 50615.1 | 32157.4 | 0.0124 | 8644.3 | 3761.3 | 0.0018 |
| AB023418 | 92459_at | 682.8 | 2312.4 | 0.0464 | 624.5 | 33115.2 | 0.0001 |
| X66032 | 94294_at | 998.2 | 1429.9 | 0.0088 | 3392.3 | 2047.4 | 0.0008 |
| AW047630 | 99535_at | 18919.9 | 10694.1 | 0.0333 | 3717.8 | 5383.2 | 0.0237 |
| AI847784 | 160358_at | 680.0 | 1124.7 | 0.0281 | 252.6 | 450.5 | 0.0295 |
| L78075 | 94105_at | 9941.6 | 7597.7 | 0.0006 | 12300.8 | 9734.5 | 0.0140 |
| M31131 | 102852_at | 80.2 | 234.1 | 0.0498 | 1749.1 | 814.7 | 0.0161 |
| AI854020 | 96346_at | 71828.6 | 45731.2 | 0.0032 | 623.5 | 2584.8 | 0.0052 |
| AV336987 | 161941_r_at | 2919.9 | 4189.9 | 0.0194 | 3509.7 | 4786.5 | 0.0204 |
| AI838398 | 96725_at | 1119.0 | 1414.1 | 0.0235 | 2275.2 | 3315.1 | 0.0079 |
| Y15163 | 101973_at | 6854.0 | 3597.3 | 0.0196 | 8237.5 | 3678.1 | 0.0014 |
| Z18272 | 93517_at | 15077.4 | 8457.4 | 0.0091 | 3323.0 | 6487.4 | 0.0035 |
| AF017175 | 93320_at | 6518.6 | 3200.5 | 0.0087 | 8312.1 | 3186.6 | 0.0001 |
| AV013428 | 162308_f_at | 1503.8 | 2208.4 | 0.0028 | 4465.9 | 2218.9 | 0.0080 |
| AI837625 | 160065_s_at | 4354.7 | 2016.4 | 0.0022 | 17483.3 | 3532.9 | 0.0000 |
| U49385 | 160652_at | 1599.6 | 1234.2 | 0.0155 | 3356.0 | 2021.9 | 0.0020 |
| U74683 | 101019_at | 2225.8 | 1023.3 | 0.0072 | 1143.9 | 251.9 | 0.0022 |
| AW061318 | 97255_at | 2692.1 | 1369.1 | 0.0436 | 3647.5 | 1727.0 | 0.0201 |
| U27267 | 98772_at | 147.8 | 43.9 | 0.0462 | 246.4 | 789.6 | 0.0131 |
| X78445 | 99979_at | 1289.4 | 117.7 | 0.0299 | 30354.7 | 15883.0 | 0.0033 |
| D63679 | 103617_at | 276.9 | 472.4 | 0.0383 | 374.1 | 608.6 | 0.0315 |
| AB026432 | 95683_g_at | 8997.6 | 7136.2 | 0.0051 | 20606.4 | 16879.8 | 0.0114 |
| Z38015 | 93431_at | 12219.9 | 9890.5 | 0.0197 | 14812.4 | 7269.5 | 0.0011 |
| AW060270 | 97868_at | 3304.7 | 4534.0 | 0.0031 | 3134.2 | 3784.0 | 0.0215 |
| M76131 | 97559_at | 21077.2 | 16056.3 | 0.0358 | 23817.2 | 20575.5 | 0.0386 |
| U57686 | 96195_at | 878.9 | 1414.4 | 0.0453 | 1206.9 | 2448.8 | 0.0009 |
| AW049716 | 101841_at | 805.7 | 380.1 | 0.0329 | 3471.5 | 5432.1 | 0.0236 |
| AJ006587 | 94252_at | 10301.5 | 7696.0 | 0.0353 | 10657.9 | 8904.5 | 0.0260 |
| X98471 | 97426_at | 50456.2 | 27341.7 | 0.0044 | 19625.7 | 9467.9 | 0.0046 |
| L12703 | 96523_at | 53.1 | 422.9 | 0.0128 | 1005.3 | 2116.3 | 0.0083 |
| M29961 | 102373_at | 6763.2 | 2915.9 | 0.0006 | 670.2 | 1147.2 | 0.0259 |
| J02700 | 104174_at | 26.6 | 93.1 | 0.0347 | 662.5 | 104.4 | 0.0062 |
| AW122933 | 97317_at | 21426.3 | 13197.2 | 0.0039 | 297.4 | 1848.3 | 0.0005 |
| AW061222 | 97517_at | 1934.3 | 2521.4 | 0.0312 | 1258.9 | 1649.7 | 0.0420 |
| U41739 | 97498_at | 16061.1 | 12045.1 | 0.0257 | 16926.6 | 1944.8 | 0.0029 |
| DI6215 | 101991_at | 9522.8 | 13037.1 | 0.0311 | 263.6 | 1443.3 | 0.0017 |
| AF017128 | 99835_at | 1185.8 | 373.0 | 0.0478 | 1467.6 | 3371.1 | 0.0025 |
| AI839918 | 93270_at | 4261.9 | 3501.9 | 0.0173 | 10705.2 | 7081.3 | 0.0335 |
| AB000096 | 102789_at | 694.4 | 1140.3 | 0.0291 | 1799.9 | 729.8 | 0.0001 |
| L41631 | 102651_at | 278.3 | 750.8 | 0.0081 | 601.3 | 830.7 | 0.0369 |
| U15012 | 99108_s_at | 26654.2 | 19789.5 | 0.0396 | 1414.8 | 2202.9 | 0.0108 |
| AI153412 | 104412_at | 8093.8 | 5438.8 | 0.0124 | 2394.2 | 625.0 | 0.0050 |
| X83577 | 102886_at | 2522.8 | 1687.2 | 0.0102 | 7312.0 | 2151.0 | 0.0090 |
| D50430 | 98984_f_at | 4526.9 | 7135.8 | 0.0331 | 851.3 | 1110.8 | 0.0061 |
| AB003502 | 93727_at | 6809.2 | 4002.5 | 0.0280 | 7801.1 | 6419.4 | 0.0229 |
| AF043220 | 94296_s_at | 2002.8 | 1504.9 | 0.0173 | 941.1 | 680.0 | 0.0237 |
| M69069 | 97540_f_at | 56632.0 | 84722.1 | 0.0060 | 19345.1 | 48703.0 | 0.0026 |
| X52490 | 101886_f_at | 47481.0 | 67094.4 | 0.0037 | 18901.1 | 50653.5 | 0.0004 |
| V00746 | 93120_f_at | 66239.3 | 96624.8 | 0.0083 | 25468.5 | 59120.4 | 0.0001 |
| X16426 | 101898_s_at | 3484.2 | 14555.1 | 0.0139 | 764.4 | 3709.8 | 0.0000 |
| Y00629 | 98472_at | 566.5 | 1182.3 | 0.0013 | 1837.9 | 3367.8 | 0.0001 |
| U05837 | 94840_at | 1452.0 | 2031.7 | 0.0150 | 2229.4 | 6725.3 | 0.0001 |
| AF077659 | 103833_at | 7260.1 | 3440.7 | 0.0113 | 428.0 | 169.9 | 0.0191 |
| Y00208 | 103086_at | 1604.8 | 837.8 | 0.0305 | 1657.3 | 242.6 | 0.0000 |
| X07439 | 93378_at | 2484.5 | 1597.2 | 0.0374 | 3351.3 | 1144.9 | 0.0003 |
| X55318 | 92891_f_at | 674.4 | 1189.3 | 0.0042 | 748.2 | 564.6 | 0.0357 |
| U44389 | 93351_at | 930.5 | 425.9 | 0.0401 | 2136.2 | 641.6 | 0.0024 |
| AI837110 | 96696_at | 1628.4 | 990.7 | 0.0287 | 3705.9 | 2379.7 | 0.0369 |
| Y15733 | 94177_at | 374.9 | 158.7 | 0.0274 | 668.6 | 461.1 | 0.0181 |
| AA762325 | 97859_at | 2627.2 | 1327.8 | 0.0086 | 5876.8 | 3991.2 | 0.0105 |
| M21065 | 102401_at | 9524.6 | 17803.2 | 0.0055 | 793.0 | 1186.8 | 0.0367 |
| Y11460 | 94826_at | 2244.9 | 1790.8 | 0.0016 | 2332.9 | 3410.1 | 0.0007 |
| M90365 | 104121_at | 3445.4 | 1685.8 | 0.0010 | 1314.7 | 447.7 | 0.0009 |
| AB013345 | 102020_at | 501.0 | 1359.0 | 0.0132 | 20.0 | 15.6 | 0.0472 |
| U36340 | 100010_at | 751.9 | 652.0 | 0.0488 | 1684.7 | 1343.9 | 0.0477 |
| AW047023 | 96010_at | 3542.9 | 2569.8 | 0.0430 | 4870.3 | 4120.3 | 0.0186 |
| AF034745 | 102038_at | 138.8 | 212.6 | 0.0336 | 117.7 | 258.7 | 0.0016 |
| M63335 | 160083_at | 59754.7 | 40987.4 | 0.0078 | 5915.5 | 11405.0 | 0.0019 |
| U27195 | 92401_at | 9509.6 | 5737.5 | 0.0051 | 632.4 | 906.3 | 0.0233 |
| D86232 | 93077_s_at | 12075.9 | 21116.2 | 0.0363 | 7720.2 | 18785.1 | 0.0008 |
| AI848045 | 93749_at | 2311.3 | 1325.5 | 0.0049 | 4965.7 | 10727.1 | 0.0004 |
| AI317205 | 103020_s_at | 1197.6 | 1453.4 | 0.0467 | 818.0 | 1057.5 | 0.0233 |
| Y13439 | 92323_at | 596.2 | 978.5 | 0.0476 | 1088.5 | 1708.2 | 0.0096 |
| AI844810 | 103416_at | 21093.8 | 11355.8 | 0.0004 | 9413.9 | 13726.7 | 0.0088 |
| AB005662 | 160880_at | 678.6 | 1004.7 | 0.0150 | 868.2 | 1431.0 | 0.0162 |
| AF072240 | 104340_at | 1723.0 | 659.4 | 0.0228 | 1693.7 | 858.1 | 0.0494 |
| AI853261 | 160458_at | 8292.3 | 5080.0 | 0.0082 | 815.0 | 325.3 | 0.0001 |
| AI841279 | 100539_at | 1566.2 | 851.8 | 0.0233 | 2153.9 | 779.6 | 0.0001 |
| X66402 | 98833_at | 179.5 | 622.3 | 0.0475 | 14273.8 | 68072.2 | 0.0006 |
| J02652 | 101082_at | 47789.2 | 63641.8 | 0.0269 | 8250.1 | 6163.1 | 0.0039 |
| AB004879 | 103653_at | 555.5 | 167.3 | 0.0132 | 951.7 | 597.1 | 0.0107 |
| AI255271 | 102096_f_at | 128.0 | 9573.7 | 0.0226 | 50.3 | 149.4 | 0.0011 |
| M16359 | 101910_f_at | 183.1 | 11635.4 | 0.0321 | 36.7 | 68.7 | 0.0407 |
| X51829 | 160463_at | 2434.6 | 3653.3 | 0.0215 | 1268.8 | 1630.9 | 0.0278 |
| AI648850 | 100828_at | 1041.0 | 1659.4 | 0.0290 | 650.4 | 1261.2 | 0.0067 |
| AI117835 | 93482_at | 13090.5 | 6705.6 | 0.0002 | 37600.0 | 7683.1 | 0.0020 |
| U96723 | 95506_at | 2031.0 | 906.5 | 0.0336 | 3157.4 | 2087.1 | 0.0244 |
| U81453 | 94713_at | 918.9 | 1198.3 | 0.0119 | 2868.2 | 3548.0 | 0.0141 |
| X61449 | 98587_at | 4299.8 | 2754.7 | 0.0286 | 7851.0 | 3334.8 | 0.0067 |
| AW125874 | 95070_at | 1143.4 | 796.7 | 0.0190 | 5282.0 | 3996.4 | 0.0017 |
| U83148 | 102955_at | 3785.4 | 1707.8 | 0.0075 | 1632.7 | 1039.5 | 0.0212 |
| Y07688 | 101930_at | 8742.6 | 6157.7 | 0.0270 | 8430.9 | 6011.9 | 0.0193 |
| AB017202 | 93563_s_at | 3536.5 | 1703.8 | 0.0051 | 29717.3 | 17043.2 | 0.0181 |
| Z49204 | 99009_at | 1892.7 | 964.1 | 0.0173 | 5358.6 | 2207.2 | 0.0003 |
| AI839690 | 103922_f_at | 864.2 | 519.2 | 0.0464 | 4941.0 | 2840.9 | 0.0113 |
| X74134 | 102715_at | 551.1 | 101.1 | 0.0057 | 1383.2 | 289.9 | 0.0004 |
| AA645293 | 97977_at | 733.1 | 1220.0 | 0.0057 | 1199.2 | 2054.2 | 0.0021 |
| AF089751 | 95586_at | 1077.8 | 1530.7 | 0.0094 | 2404.7 | 4921.1 | 0.0000 |
| AJ009823 | 101712_at | 997.9 | 1810.0 | 0.0270 | 793.7 | 1259.4 | 0.0080 |
| AI846025 | 95470_at | 4077.9 | 2298.9 | 0.0217 | 1407.0 | 1702.8 | 0.0441 |
| AB006758 | 102280_at | 756.2 | 294.5 | 0.0182 | 1373.0 | 497.7 | 0.0009 |
| X57337 | 93349_at | 1721.3 | 4735.5 | 0.0042 | 23473.4 | 47164.8 | 0.0042 |
| D50060 | 101196_at | 879.6 | 1671.0 | 0.0052 | 4967.6 | 1661.0 | 0.0063 |
| AA755004 | 96831_at | 63.5 | 374.9 | 0.0161 | 1939.4 | 5034.1 | 0.0004 |
| AI842259 | 92810_at | 966.4 | 385.0 | 0.0178 | 6964.0 | 3660.8 | 0.0010 |
| AF053367 | 100554_at | 1740.5 | 1093.8 | 0.0134 | 10364.4 | 5104.7 | 0.0130 |
| U44088 | 160829_at | 8260.0 | 13438.1 | 0.0111 | 6162.8 | 10902.3 | 0.0025 |
| AA607557 | 161034_at | 284.8 | 746.2 | 0.0205 | 475.5 | 656.6 | 0.0177 |
| U85711 | 104580_at | 1760.7 | 893.9 | 0.0033 | 1997.4 | 1423.8 | 0.0040 |
| AW047139 | 96774_at | 2483.6 | 1584.8 | 0.0376 | 5635.3 | 4697.2 | 0.0109 |
| Z38110 | 102395_at | 14731.0 | 9813.7 | 0.0169 | 33630.2 | 18940.4 | 0.0002 |
| AW123013 | 99183_at | 7021.4 | 5762.2 | 0.0129 | 6489.0 | 4811.7 | 0.0122 |
| AF093857 | 100622_at | 19772.8 | 10598.1 | 0.0067 | 15049.0 | 28212.0 | 0.0003 |
| AF093853 | 100332_s_at | 9035.9 | 4555.0 | 0.0091 | 9217.0 | 19732.5 | 0.0012 |
| AW122197 | 96852_at | 9116.7 | 6581.3 | 0.0162 | 16038.7 | 13215.3 | 0.0216 |
| AV353694 | 161446_r_at | 5368.4 | 8850.8 | 0.0445 | 6341.9 | 7869.5 | 0.0091 |
| AW122030 | 96295_at | 14317.5 | 8059.2 | 0.0050 | 11078.7 | 7195.3 | 0.0019 |
| U22033 | 102791_at | 1464.2 | 4114.7 | 0.0102 | 1964.7 | 3551.1 | 0.0009 |
| D44456 | 93085_at | 923.8 | 3031.4 | 0.0156 | 280.9 | 1003.0 | 0.0115 |
| AI845915 | 104100_at | 43869.2 | 36308.4 | 0.0448 | 9141.5 | 4899.8 | 0.0138 |
| M89777 | 97415_at | 4734.7 | 3200.4 | 0.0127 | 1449.5 | 1776.4 | 0.0455 |
| X89650 | 99587_at | 3208.4 | 2386.6 | 0.0365 | 3173.9 | 2756.2 | 0.0164 |
| AI844445 | 102117_at | 1345.9 | 594.0 | 0.0348 | 1178.8 | 632.5 | 0.0011 |
| X57277 | 101555_at | 31407.3 | 18445.6 | 0.0158 | 42855.4 | 31108.0 | 0.0023 |
| D64162 | 102649_s_at | 104.9 | 464.9 | 0.0022 | 1754.7 | 829.2 | 0.0101 |
| AI847564 | 93070_at | 1324.4 | 957.6 | 0.0436 | 3290.6 | 2617.0 | 0.0430 |
| AB016424 | 96041_at | 7605.2 | 3592.8 | 0.0027 | 10082.0 | 7170.8 | 0.0128 |
| AW046449 | 96207_at | 2836.5 | 1945.1 | 0.0249 | 13237.0 | 7607.3 | 0.0141 |
| AI048434 | 160518_at | 5922.5 | 4298.2 | 0.0188 | 4844.0 | 4013.7 | 0.0485 |
| AF014371 | 101112_g_at | 14325.1 | 10492.6 | 0.0392 | 13158.0 | 9154.3 | 0.0131 |
| AW121012 | 100509_at | 2024.6 | 1360.7 | 0.0215 | 3508.4 | 2112.6 | 0.0082 |
| U58513 | 98504_at | 2947.2 | 1543.6 | 0.0085 | 11648.0 | 6191.0 | 0.0076 |
| M83218 | 103448_at | 1075.6 | 480.1 | 0.0009 | 272.8 | 492.9 | 0.0478 |
| X03505 | 102712_at | 13920.3 | 5441.6 | 0.0300 | 61929.6 | 102890.2 | 0.0061 |
| L10244 | 96657_at | 9701.0 | 6761.9 | 0.0170 | 7749.4 | 17947.1 | 0.0008 |
| AB008553 | 101389_at | 3358.1 | 4064.3 | 0.0402 | 14319.9 | 9631.1 | 0.0220 |
| M21285 | 94057_g_at | 231696.5 | 305128.6 | 0.0144 | 7982.8 | 19207.9 | 0.0023 |
| U88567 | 93503_at | 281.2 | 1510.6 | 0.0405 | 621.2 | 6317.6 | 0.0000 |
| U66918 | 99042_s_at | 282.1 | 2457.0 | 0.0004 | 397.2 | 3622.3 | 0.0023 |
| AJ243651 | 100373_at | 166.5 | 311.5 | 0.0371 | 131.9 | 176.4 | 0.0347 |
| AF004666 | 99524_at | 611.6 | 265.7 | 0.0100 | 3396.8 | 996.2 | 0.0041 |
| AI839882 | 94034_at | 2871.2 | 1875.4 | 0.0201 | 4247.6 | 1982.7 | 0.0009 |
| U88328 | 92232_at | 3605.6 | 17509.2 | 0.0013 | 1969.1 | 4618.8 | 0.0094 |
| AJ005567 | 95794_f_at | 1233.3 | 2070.9 | 0.0067 | 3158.0 | 5385.1 | 0.0302 |
| AI837107 | 103504_at | 1038.7 | 1226.3 | 0.0056 | 2045.5 | 1207.7 | 0.0052 |
| U47323 | 100952_at | 9903.8 | 3763.6 | 0.0424 | 1916.9 | 1387.8 | 0.0283 |
| AI842665 | 93327_at | 2700.0 | 1935.9 | 0.0180 | 2745.0 | 2191.4 | 0.0287 |
| AF041822 | 102256_at | 20.8 | 255.0 | 0.0012 | 23.1 | 727.1 | 0.0124 |
| U86137 | 93367_at | 755.9 | 153.3 | 0.0140 | 628.4 | 495.9 | 0.0425 |
| L19932 | 92877_at | 501.6 | 735.6 | 0.0447 | 93.2 | 353.1 | 0.0137 |
| X14432 | 104601_at | 2245.4 | 861.4 | 0.0094 | 5919.2 | 1878.5 | 0.0005 |
| M62470 | 160469_at | 8011.5 | 1387.4 | 0.0209 | 41141.3 | 26162.1 | 0.0052 |
| AI849587 | 95465_s_at | 743.1 | 1129.6 | 0.0122 | 2896.5 | 1034.0 | 0.0491 |
| AI852433 | 104071_at | 3352.1 | 2358.6 | 0.0131 | 5282.7 | 3615.1 | 0.0487 |
| L31777 | 99566_at | 27393.0 | 23196.3 | 0.0026 | 27201.0 | 16484.4 | 0.0005 |
| M28729 | 100343_f_at | 94370.3 | 50995.0 | 0.0069 | 38286.9 | 19310.8 | 0.0287 |
| M13441 | 101543_f_at | 160632.1 | 76299.2 | 0.0013 | 59543.9 | 34148.4 | 0.0377 |
| M28739 | 94835_f_at | 7670.9 | 4789.0 | 0.0080 | 17788.7 | 9599.6 | 0.0037 |
| X04663 | 94788_f_at | 18550.8 | 11564.0 | 0.0173 | 28376.4 | 17148.7 | 0.0001 |
| AI840882 | 95696_at | 21807.0 | 14814.2 | 0.0322 | 10205.9 | 7138.5 | 0.0036 |
| AW046479 | 102279_at | 643.6 | 1293.8 | 0.0141 | 1376.1 | 2043.3 | 0.0097 |
| AB001489 | 99926_at | 1009.4 | 1814.9 | 0.0344 | 1320.4 | 1793.4 | 0.0218 |
| AB010742 | 93392_at | 827.0 | 1913.7 | 0.0034 | 152.7 | 232.2 | 0.0222 |
| AF026469 | 99064_at | 4105.2 | 3382.3 | 0.0295 | 4249.9 | 3171.4 | 0.0029 |
| AI847972 | 98521_at | 3108.4 | 2425.5 | 0.0290 | 3010.6 | 2311.7 | 0.0101 |
| AI462105 | 94963_at | 13498.7 | 7326.6 | 0.0458 | 34083.9 | 22131.3 | 0.0132 |
| AW061016 | 99964_at | 27.0 | 53.5 | 0.0144 | 439.6 | 40.2 | 0.0278 |
| X69656 | 98606_s_at | 1296.5 | 733.9 | 0.0416 | 1631.7 | 944.0 | 0.0183 |
| D87661 | 97535_at | 3854.3 | 2559.2 | 0.0014 | 7650.0 | 6463.3 | 0.0086 |

| Gene ID | Gene Title | Gene Symbol | PpFDRst |
|---|---|---|---|
| AE000663 | RIKEN cDNA 1810009J06 gene | 1810009J06Rik | 0.005 |
| AA675604 | RIKEN cDNA 4930517K11 gene | 4930517K11Rik | 0.003 |
| AI849271 | acetyl-Coenzyme A acyltransferase | Acaa2 | 0.004 |
| | 2 (mitochondrial 3-oxoacyl- | | |
| | Coenzyme A thiolase) | | |
| J04946 | angiotensin converting enzyme | Ace | 0.003 |
| AI838021 | acyl-CoA synthetase long-chain | Acsl5 | 0.003 |
| | family member 5 | | |
| AF045887 | angiotensinogen | Agt | 0.003 |
| AB027125 | aldo-keto reductase family 1, | Akr1c12 | 0.003 |
| | member C12 | | |
| M74570 | aldehyde dehydrogenase family 1, | Aldh1a1 | 0.003 |
| | subfamily A1 | | |
| AW123269 | anillin, actin binding protein (scraps | Anln | 0.003 |
| | homolog, Drosophila) | | |
| AV003419 | annexin A1 | Anxa1 | 0.003 |
| M14044 | annexin A2 | Anxa2 | 0.005 |
| AJ002390 | annexin A8 | Anxa8 | 0.004 |
| M97216 | amyloid beta (A4) precursor-like | Aplp2 | 0.004 |
| | protein 2 | | |
| X82648 | apolipoprotein D | Apod | 0.008 |
| D87901 | ADP-ribosylation factor 4 | Arf4 | 0.004 |
| AI852332 | ADP-ribosylation factor interacting | Arfip2 | 0.004 |
| | protein 2 | | |
| AI846773 | Rho GTPase activating protein 1 | Arhgap1 | 0.003 |
| M63725 | activating transcription factor 1 | Atf1 | 0.033 |
| U13840 | ATPase, H+ transporting, V0 | Atp6v0d1 | 0.004 |
| | subunit D isoform 1 | | |
| X01838 | beta-2 microglobulin | B2m | 0.003 |
| X55573 | brain derived neurotrophic factor | Bdnf | 0.003 |
| D83745 | B-cell translocation gene 3 | Btg3 | 0.005 |
| X06454 | complement component 4 (within | C4///Slp | 0.003 |
| | H-2S)///sex-limited protein | | |
| M19381 | calmodulin 1 | Calm1 | 0.005 |
| M27844 | calmodulin 2 | Calm2 | 0.004 |
| AI842328 | calmodulin 3 | Calm3 | 0.006 |
| U16740 | capping protein (actin filament) | Capza1 | 0.007 |
| | muscle Z-line, alpha 1 | | |
| U16741 | Capping protein (actin filament) | Capza2 | 0.004 |
| | muscle Z-line, alpha 2 | | |
| AI747654 | caveolin, caveolae protein 1 | Cav1 | 0.003 |
| AB023418 | chemokine (C-C motif) ligand 8 | Ccl8 | 0.003 |
| X66032 | cyclin B2 | Ccnb2 | 0.009 |
| AW047630 | CCR4 carbon catabolite repression | Ccrn4l | 0.025 |
| | 4-like (S. cerevisiae) | | |
| AI847784 | CD34 antigen | Cd34 | 0.015 |
| L78075 | cell division cycle 42 homolog (S. | Cdc42 | 0.003 |
| | cerevisiae) | | |
| M31131 | cadherin 2 | Cdh2 | 0.005 |
| AI854020 | cysteine dioxygenase 1, cytosolic | Cdo1 | 0.004 |
| AV336987 | Centaurin, gamma 3 | Centg3 | 0.008 |
| AI838398 | capicua homolog (Drosophila) | Cic | 0.005 |
| Y15163 | Cbp/p300-interacting transactivator, | Cited2 | 0.003 |
| | with Glu/Asp-rich carboxy-terminal | | |
| | domain, 2 | | |
| Z18272 | procollagen, type VI, alpha 2 | Col6a2 | 0.032 |
| AF017175 | camitine palmitoyl transferase 1a, | Cpt1a | 0.003 |
| | liver | | |
| AV013428 | crystallin, alpha B | Cryab | 0.010 |
| AI837625 | cysteine and glycine-rich protein 1 | Csrp1 | 0.003 |
| U49385 | cytidine 5′-triphosphate synthase 2 | Ctps2 | 0.003 |
| U74683 | cathepsin C | Ctsc | 0.003 |
| AW061318 | CUG triplet repeat, RNA binding | Cugbp2 | 0.010 |
| | protein 2 | | |
| U27267 | chemokine (C-X-C motif) ligand 5 | Cxcl5 | 0.005 |
| X78445 | cytochrome P450, family 1, | Cyp1b1 | 0.003 |
| | subfamily b, polypeptide 1 | | |
| D63679 | decay accelerating factor 1 | Daf1 | 0.016 |
| AB026432 | damage specific DNA binding | Ddb1 | 0.005 |
| | protein 1 | | |
| Z38015 | dystrophia myotonica-protein | Dmpk | 0.003 |
| | kinase | | |
| AW060270 | DnaJ (Hsp40) homolog, subfamily | Dnaja3 | 0.004 |
| | A, member 3 | | |
| M76131 | eukaryotic translation elongation | Eef2 | 0.007 |
| | factor 2 | | |
| U57686 | embryonal Fyn-associated substrate | Efs | 0.004 |
| AW049716 | epidermal growth factor receptor | Egfr | 0.008 |
| AJ006587 | eukaryotic translation initiation | Eif2s3x | 0.012 |
| | factor 2, subunit 3, structural gene | | |
| | X-linked | | |
| X98471 | epithelial membrane protein 1 | Emp1 | 0.004 |
| L12703 | engrailed 1 | En1 | 0.003 |
| M29961 | glutamyl aminopeptidase | Enpep | 0.003 |
| J02700 | ectonucleotide | Enpp1 | 0.003 |
| | pyrophosphatase/phosphodiesterase 1 | | |
| AW122933 | ectonucleotide | Enpp2 | 0.004 |
| | pyrophosphatase/phosphodiesterase 2 | | |
| AW061222 | exosome component 4 | Exosc4 | 0.014 |
| U41739 | four and a half LIM domains 1 | Fhl1 | 0.003 |
| DI6215 | flavin containing monooxygenase 1 | Fmo1 | 0.005 |
| AF017128 | fos-like antigen 1 | Fosl1 | 0.020 |
| AI839918 | glycyl-tRNA synthetase | Gars | 0.006 |
| AB000096 | GATA binding protein 2 | Gata2 | 0.012 |
| L41631 | glucokinase | Gck | 0.005 |
| U15012 | growth hormone receptor | Ghr | 0.016 |
| AI153412 | guanine nucleotide binding protein, | Gnai1 | 0.003 |
| | alpha inhibiting 1 | | |
| X83577 | glypican 4 | Gpc4 | 0.003 |
| D50430 | glycerol phosphate dehydrogenase | Gpd2 | 0.010 |
| | 2, mitochondrial | | |
| AB003502 | G1 to S phase transition 1 | Gspt1 | 0.007 |
| AF043220 | general transcription factor II I | Gtf2i | 0.010 |
| M69069 | histocompatibility 2, D region locus | H2-D1 | 0.003 |
| | 1 | | |
| X52490 | histocompatibility 2, D region locus | H2-D1///H2-L | 0.003 |
| | 1///histocompatibility 2, D region | | |
| V00746 | histocompatibility 2, K1, K region | H2-K1 | 0.003 |
| X16426 | histocompatibility 2, Q region locus | H2-Q10 | 0.003 |
| | 10 | | |
| Y00629 | histocompatibility 2, T region locus | H2-T23 | 0.003 |
| | 23 | | |
| U05837 | hexosaminidase A | Hexa | 0.003 |
| AF077659 | homeodomain interacting protein | Hipk2 | 0.007 |
| | kinase 2 | | |
| Y00208 | Homeobox A5 | Hoxa5 | 0.003 |
| X07439 | Homeobox C8 | Hoxc8 | 0.003 |
| X55318 | Homeobox C9 | Hoxc9 | 0.047 |
| U44389 | hydroxyprostaglandin | Hpgd | 0.003 |
| | dehydrogenase 15 (NAD) | | |
| AI837110 | heterogeneous nuclear | Hrmt1l2 | 0.007 |
| | ribonucleoproteins | | |
| | methyltransferase-like 2 (S. | | |
| | cerevisiae) | | |
| Y15733 | hydroxysteroid (17-beta) | Hsd17b7 | 0.007 |
| | dehydrogenase 7 | | |
| AA762325 | inositol polyphosphate-5- | Inpp5a | 0.003 |
| | phosphatase A | | |
| M21065 | interferon regulatory factor 1 | Irf1 | 0.005 |
| Y11460 | integrin beta 4 binding protein | Itgb4bp | 0.008 |
| M90365 | junction plakoglobin | Jup | 0.003 |
| AB013345 | potassium channel, subfamily K, | Kcnk3 | 0.003 |
| | member 3 | | |
| U36340 | Kruppel-like factor 3 (basic) | Klf3 | 0.024 |
| AW047023 | karyopherin (importin) alpha 3 | Kpna3 | 0.016 |
| AF034745 | ligand of numb-protein X 1 | Lnx1 | 0.004 |
| M63335 | lipoprotein lipase | Lpl | 0.004 |
| U27195 | leukotriene C4 synthase | Ltc4s | 0.006 |
| D86232 | lymphocyte antigen 6 complex, | Ly6c | 0.003 |
| | locus C | | |
| AI848045 | monoamine oxidase A | Maoa | 0.003 |
| AI317205 | mitogen activated protein kinase | Map3k1 | 0.010 |
| | kinase kinase 1 | | |
| Y13439 | mitogen-activated protein kinase 12 | Mapk12 | 0.009 |
| AI844810 | mitogen-activated protein kinase 6 | Mapk6 | 0.016 |
| AB005662 | mitogen-activated protein kinase 8 | Mapk8ip3 | 0.005 |
| | interacting protein 3 | | |
| AF072240 | methyl-CpG binding domain | Mbd1 | 0.004 |
| | protein 1 | | |
| AI853261 | melanoma cell adhesion molecule | Mcam | 0.003 |
| AI841279 | brain acyl-CoA hydrolase | MGI: 1917275 | 0.003 |
| X66402 | matrix metalloproteinase 3 | Mmp3 | 0.003 |
| J02652 | malic enzyme, supernatant | Mod1 | 0.018 |
| AB004879 | muscle and microspikes RAS | Mras | 0.004 |
| AI255271 | major urinary protein 1///major | Mup1///Mup2/// | 0.003 |
| | urinary protein 2///major urinary | Mup3///Mup4/// | |
| | protein 3///major urinary protein 4/// | Mup5 | |
| | major urinary protein 5 | | |
| M16359 | major urinary protein 3 | Mup3 | 0.003 |
| X51829 | myeloid differentiation primary | Myd116 | 0.012 |
| | response gene 116 | | |
| AI648850 | myosin, light polypeptide 4 | Myl4 | 0.008 |
| AI117835 | myosin, light polypeptide kinase | Mylk | 0.003 |
| U96723 | myosin IC | Myo1c | 0.009 |
| U81453 | myosin VIIa | Myo7a | 0.005 |
| X61449 | nucleosome assembly protein 1-like 1 | Nap1l1 | 0.003 |
| AW125874 | asparaginyl-tRNA synthetase | Nars | 0.003 |
| U83148 | nuclear factor, interleukin 3, | Nfil3 | 0.005 |
| | regulated | | |
| Y07688 | nuclear factor I/X | Nfix | 0.006 |
| AB017202 | nidogen 2 | Nid2 | 0.005 |
| Z49204 | nicotinamide nucleotide | Nnt | 0.003 |
| | transhydrogenase | | |
| AI839690 | NAD(P)H: quinone oxidoreductase | Nqo3a2 | 0.007 |
| | type 3, polypeptide A2 | | |
| X74134 | nuclear receptor subfamily 2, group | Nr2f1 | 0.003 |
| | F, member 1 | | |
| AA645293 | netrin 1 | Ntn1 | 0.003 |
| AF089751 | purinergic receptor P2X, ligand- | P2rx4 | 0.003 |
| | gated ion channel 4 | | |
| AJ009823 | purinergic receptor P2X, ligand- | P2rx7 | 0.009 |
| | gated ion channel, 7 | | |
| AI846025 | PAK1 interacting protein 1 | Pak1ip1 | 0.009 |
| AB006758 | protocadherin 7 | Pcdh7 | 0.003 |
| X57337 | procollagen C-proteinase enhancer | Pcolce | 0.003 |
| | protein | | |
| D50060 | proprotein convertase | Pcsk6 | 0.004 |
| | subtilisin/kexin type 6 | | |
| AA755004 | protein disulfide isomerase | Pdia5 | 0.003 |
| | associated 5 | | |
| AI842259 | pyruvate dehydrogenase kinase, | Pdk3 | 0.003 |
| | isoenzyme 3 | | |
| AF053367 | PDZ and LIM domain 1 (elfin) | Pdlim1 | 0.003 |
| U44088 | pleckstrin homology-like domain, | Phlda1 | 0.003 |
| | family A, member 1 | | |
| AA607557 | phospholipase A2, group X | Pla2g10 | 0.007 |
| U85711 | phospholipase C, delta 1 | Plcd1 | 0.003 |
| AW047139 | pleckstrin homology domain | Plekhc1 | 0.005 |
| | containing, family C (with FERM | | |
| | domain) member 1 | | |
| Z38110 | peripheral myelin protein | Pmp22 | 0.003 |
| AW123013 | Protein phosphatase 3, regulatory | Ppp3r1 | 0.006 |
| | subunit B, alpha isoform | | |
| | (calcineurin B, type I) | | |
| AF093857 | peroxiredoxin 6 | Prdx6 | 0.045 |
| AF093853 | peroxiredoxin 6///peroxiredoxin 6, | Prdx6///Prdx6- | 0.006 |
| | related sequence 1 | rs1 | |
| AW122197 | protein kinase, cAMP dependent | Prkar1a | 0.004 |
| | regulatory, type I, alpha | | |
| AV353694 | protease, serine, 25 | Prss25 | 0.013 |
| AW122030 | phosphoserine aminotransferase 1 | Psat1 | 0.003 |
| U22033 | proteosome (prosome, macropain) | Psmb8 | 0.003 |
| | subunit, beta type 8 (large | | |
| | multifunctional protease 7) | | |
| D44456 | proteosome (prosome, macropain) | Psmb9 | 0.003 |
| | subunit, beta type 9 (large | | |
| | multifunctional protease 2) | | |
| AI845915 | polymerase I and transcript release | Ptrf | 0.008 |
| | factor | | |
| M89777 | RAB3D, member RAS oncogene | Rab3d | 0.022 |
| | family | | |
| X89650 | RAB7, member RAS oncogene | Rab7 | 0.007 |
| | family | | |
| AI844445 | RAB, member of RAS oncogene | Rabl4 | 0.003 |
| | family-like 4 | | |
| X57277 | RAS-related C3 botulinum substrate 1 | Rac1 | 0.004 |
| D64162 | retinoic acid early transcript 1, | Raet1a///Raet1b | 0.007 |
| | alpha///retinoic acid early | ///Raet1c/// | |
| | transcript beta///retinoic acid early | Raet1d///Raet1e | |
| | transcript gamma///retinoic acid | | |
| | early transcript delta///retinoic acid | | |
| | early transcript 1E | | |
| AI847564 | RAN binding protein 5 | Ranbp5 | 0.007 |
| AB016424 | RNA binding motif protein 3 | Rbm3 | 0.004 |
| AW046449 | RNA binding motif, single stranded | Rbms1 | 0.005 |
| | interacting protein 1 | | |
| AI048434 | RER1 retention in endoplasmic | Rer1 | 0.013 |
| | reticulum 1 homolog (S. cerevisiae) | | |
| AF014371 | ras homolog gene family, member A | Rhoa | 0.006 |
| AW121012 | ring finger protein (C3HC4 type) 19 | Rnf19 | 0.006 |
| U58513 | Rho-associated coiled-coil forming | Rock2 | 0.003 |
| | kinase 2 | | |
| M83218 | S100 calcium binding protein A8 | S100a8 | 0.007 |
| | (calgranulin A) | | |
| X03505 | serum amyloid A 3 | Saa3 | 0.008 |
| L10244 | spermidine/spermine N1-acetyl | Sat1 | 0.008 |
| | transferase 1 | | |
| AB008553 | scavenger receptor class B, member | Scarb2 | 0.012 |
| | 2 | | |
| M21285 | stearoyl-Coenzyme A desaturase 1 | Scd1 | 0.006 |
| U88567 | secreted frizzled-related sequence | Sfrp2 | 0.003 |
| | protein 2 | | |
| U66918 | short stature Homeobox 2 | Shox2 | 0.003 |
| AJ243651 | solute carrier family 39 (zinc | Slc39a1 | 0.023 |
| | transporter), member 1 | | |
| AF004666 | solute carrier family 8 | Slc8a1 | 0.003 |
| | (sodium/calcium exchanger), | | |
| | member 1 | | |
| AI839882 | small fragment nuclease | Smfn | 0.003 |
| U88328 | suppressor of cytokine signaling 3 | Socs3 | 0.003 |
| AJ005567 | small proline-rich protein 2I | Sprr2i | 0.006 |
| AI837107 | single-stranded DNA binding | Ssbp2 | 0.005 |
| | protein 2 | | |
| U47323 | stromal interaction molecule 1 | Stim1 | 0.008 |
| AI842665 | Tax1 (human T-cell leukemia virus | Tax1bp3 | 0.008 |
| | type I) binding protein 3 | | |
| AF041822 | T-box 15 | Tbx15 | 0.003 |
| U86137 | telomerase associated protein 1 | Tep1 | 0.003 |
| L19932 | transforming growth factor, beta | Tgfbi | 0.013 |
| | induced | | |
| X14432 | thrombomodulin | Thbd | 0.003 |
| M62470 | thrombospondin 1 | Thbs1 | 0.004 |
| AI849587 | transmembrane protein 37 | Tmem37 | 0.017 |
| AI852433 | transportin 2 (importin 3, | Tnpo2 | 0.008 |
| | karyopherin beta 2b) | | |
| L31777 | triosephosphate isomerase 1 | Tpi1 | 0.003 |
| M28729 | tubulin, alpha 1 | Tuba1 | 0.004 |
| M13441 | tubulin, alpha 6 | Tuba6 | 0.003 |
| M28739 | tubulin, beta 2 | Tubb2 | 0.003 |
| X04663 | tubulin, beta 5 | Tubb5 | 0.003 |
| AI840882 | thioredoxin-like 2 | Txnl2 | 0.012 |
| AW046479 | ubiquitin-activating enzyme E1-like | Ube1l | 0.005 |
| AB001489 | upstream binding transcription | Ubtf | 0.008 |
| | factor, RNA polymerase I | | |
| AB010742 | uncoupling protein 3 | Ucp3 | 0.003 |
| | (mitochondrial, proton carrier) | | |
| AF026469 | ubiquitin specific protease 4 (proto- | Usp4 | 0.005 |
| | oncogene) | | |
| AI847972 | vesicle-associated membrane | Vamp3 | 0.005 |
| | protein 3 | | |
| AI462105 | vinculin | Vcl | 0.007 |
| AW061016 | vitamin D receptor | Vdr | 0.004 |
| X69656 | tryptophanyl-tRNA synthetase | Wars | 0.005 |
| D87661 | tyrosine 3- | Ywhah/// | 0.003 |
| | monooxygenase/tryptophan 5- | LOC545556 | |
| | monooxygenase activation protein, | | |
| | eta polypeptide///similar to 14-3-3 | | |
| | protein eta | | |

Epididymal isolated adipocytes: Ae; subcutaneous isolated adipocytes: Asc; epidydimal stromovascular fraction: Se; subcutaneous stromovascular fraction: Ssc

TABLE 2
Genes Involved in Embryonic Development, Pattern Specification, Mesoderm Formation and Organogenesis (198 Genes).

| Public ID | Gene Title | Gene Symbol | Probesets |
|---|---|---|---|
| L15436 | Activin A receptor, type 1 | Acvr1 | 93460_at |
| Z31663 | Activin A receptor, type 1B | Acvr1b | 101177_at |
| M84120 | Activin receptor IIB | Acvr2b | 93903_at |
| X99273 | Aldehyde dehydrogenase family 1, subfamily A2 | Aldh1a2 | 101707_at |
| AI854771 | Angiomotin | Amot | 95531_at |
| M88127 | Adenomatosis polyposis coli | Apc | 101447_at |
| M37890 | Androgen receptor | Ar | 92667_at |
| U77628 | Achaete-scute complex homolog-like 2 (Drosophila) | Ascl2 | 101355_at |
| AB013819 | Baculoviral IAP repeat-containing 5 | Birc5 | 101521_at |
| AA518586 | Bone morphogenetic protein 1 | Bmp1 | 92701_at, 95557_at |
| L25602 | Bone morphogenetic protein 2 | Bmp2 | 102559_at, 161118_r_at |
| X56848 | Bone morphogenetic protein 4 | Bmp4 | 93455_s_at, 93456_r_at |
| L41145 | Bone morphogenetic protein 5 | Bmp5 | 99393_at |
| X56906 | Bone morphogenetic protein 7 | Bmp7 | 93243_at |
| D16250 | Bone morphogenetic protein receptor, type 1A | Bmpr1a | 92767_at |
| Z23143 | Bone morphogenetic protein receptor, type 1B | Bmpr1b | 97725_at |
| AF003942 | Bone morphogenic protein receptor, type II (serine/threonine | Bmpr2 | 99865_at |
| AF012104 | BMX non-receptor tyrosine kinase | Bmx | 98840_at |
| M64292 | B-cell translocation gene 2, anti-proliferative | Btg2 | 101583_at |
| M80463 | Caudal type Homeobox 1 | Cdx1 | 103477_at |
| U00454 | Caudal type Homeobox 2 | Cdx2 | 103239_at |
| AI851751 | Chromodomain helicase DNA binding protein 8 | Chd8 | 104059_at, 99821_at |
| AF069501 | Chordin | Chrd | 103249_at |
| AF071313 | COP9 (constitutive photomorphogenic) homolog, subunit 3 | Cops3 | 99113_at |
| M90364 | Catenin (cadherin associated protein), beta 1, 88 kda | Ctnnb1 | 160430_at |
| AI851990 | Catenin beta interacting protein 1 | Ctnnbip1 | 99492_at |
| AW123921 | Disabled homolog 2 (Drosophila) | Dab2 | 104633_at, 98044_at, 98045_s_at |
| AV238668 | Desert hedgehog | Dhh | 161111_f_at, 161588_r_at, |
| X86925 | E2F transcription factor 5 | E2f5 | 98995_at |
| X76858 | E4F transcription factor 1 | E4f1 | 104689_at |
| U35233 | Endothelin 1 | Edn1 | 102737_at, 102738_s_at |
| U07602 | Ephrin B1 | Efnb1 | 98407_at |
| U30244 | Ephrin B2 | Efnb2 | 160857_at |
| AF016294 | E74-like factor 3 | Elf3 | 99059_at |
| L12703 | Engrailed 1 | En1 | 96523_at |
| Y00203 | Engrailed 2 | En2 | 98338_at |
| L25890 | Eph receptor B2 | Ephb2 | 98771_at |
| X96639 | Exostoses (multiple) 1 | Ext1 | 102811_at |
| U61110 | Eyes absent 1 homolog (Drosophila) | Eya1 | 94705_at, 94706_s_at |
| D89080 | Fibroblast growth factor 10 | Fgf10 | 95976_at |
| D12483 | Fibroblast growth factor 8 | Fgf8 | 97742_s_at |
| AF030635 | FK506 binding protein 8 | Fkbp8 | 100613_at |
| L35949 | Forkhead box f1a | Foxf1a | 93704_at |
| U36760 | Forkhead box G1 | Foxg1 | 161049_at |
| AF069303 | Forkhead box H1 | Foxh1 | 97789_at |
| L13204 | Forkhead box J1 | Foxj1 | 98831_at |
| X92498 | Forkhead box L1 | Foxl1 | 101185_at |
| U68058 | Frizzled-related protein | Frzb | 104672_at |
| Z29532 | Follistatin | Fst | 98817_at |
| AF054623 | Frizzled homolog 1 (Drosophila) | Fzd1 | 161040_at |
| AW123618 | Frizzled homolog 2 (Drosophila) | Fzd2 | 93681_at |
| AU020229 | Frizzled homolog 3 (Drosophila) | Fzd3 | 98169_s_at, 98348_at |
| AW122897 | Frizzled homolog 4 (Drosophila) | Fzd4 | 93459_s_at, 95771_i_at, |
| U43319 | Frizzled homolog 6 (Drosophila) | Fzd6 | 101142_at |
| U43320 | Frizzled homolog 7 (Drosophila) | Fzd7 | 101143_at |
| U43321 | Frizzled homolog 8 (Drosophila) | Fzd8 | 99415_at |
| Y17709 | Frizzled homolog 9 (Drosophila) | Fzd9 | 99844_at |
| D88611 | Glial cells missing homolog 2 (Drosophila) | Gcm2 | 94709_at, 94710_g_at |
| AF100906 | Growth differentiation factor 11 | Gdf11 | 101814_at |
| M63801 | Gap junction membrane channel protein alpha 1 | Gja1 | 100064_f_at, 100065_r_at |
| X61675 | Gap junction membrane channel protein alpha 5 | Gja5 | 101778_at |
| X95255 | GLI-Kruppel family member GLI3 | Gli3 | 101182_at |
| AA681520 | Geminin | Gmnn | 160069_at |
| AI843313 | Glypican 3 | Gpc3 | 160158_at |
| X83577 | Glypican 4 | Gpc4 | 102886_at |
| AF045801 | Gremlin 1 | Grem1 | 101758_at |
| AB017132 | Hematopoietically expressed Homeobox | Hhex | 98408_at |
| M62766 | 3-hydroxy-3-methylglutaryl-Coenzyme A reductase | Hmgcr | 104285_at, 99425_at |
| M22115 | Homeobox A1 | Hoxa1 | 95297_at |
| L08757 | Homeobox A10 | Hoxa10 | 92970_at |
| U20371 | Homeobox A11 | Hoxa11 | 104021_at |
| U59322 | Homeobox A13 | Hoxa13 | 94636_at |
| M93148 | Homeobox A2 | Hoxa2 | 102643_at |
| Y11717 | Homeobox A3 | Hoxa3 | 102087_at |
| AV279579 | Homeobox A4 | Hoxa4 | 162402_r_at |
| Y00208 | Homeobox A5 | Hoxa5 | 103086_at, 97745_at, 97746_f_at |
| M11988 | Homeobox A6 | Hoxa6 | 102579_f_at |
| M17192 | Homeobox A7 | Hoxa7 | 102864_at, 102580_r_at |
| AB005458 | Homeobox A9 | Hoxa9 | 92745_at |
| X53063 | Homeobox B1 | Hoxb1 | 93888_at |
| U57051 | Homeobox B13 | Hoxb13 | 99808_at |
| U02278 | Homeobox B3 | Hoxb3 | 98780_at |
| M36654 | Homeobox B4 | Hoxb4 | 92255_at |
| M26283 | Homeobox B5 | Hoxb5 | 103666_at |
| M18401 | Homeobox B6 | Hoxb6 | 103445_at |
| M18400 | Homeobox B7 | Hoxb7 | 92914_at |
| M18399 | Homeobox B8 | Hoxb8 | 96417_s_at |
| M34857 | Homeobox B9 | Hoxb9 | 103952_at |
| X69019 | Homeobox C4 | Hoxc4 | 102660_at |
| U28071 | Homeobox C5 | Hoxc5 | 95312_at |
| M35986 | Homeobox C6 | Hoxc6 | 99980_at |
| X07439 | Homeobox C8 | Hoxc8 | 93378_at |
| X55318 | Homeobox C9 | Hoxc9 | 92891_f_at |
| M87802 | Homeobox D1 | Hoxd1 | 98819_at |
| X62669 | Homeobox D10 | Hoxd10 | 103741_at, 98820_g_at |
| X58849 | Homeobox D12 | Hoxd12 | 99427_at |
| X99291 | Homeobox D13 | Hoxd13 | 102567_at |
| X73572 | Homeobox D3 | Hoxd3 | 98367_at |
| U77364 | Homeobox D4 | Hoxd4 | 102380_s_at |
| AI837887 | Homeobox D8 | Hoxd8 | 160460_at |
| X62669 | Homeobox D9 | Hoxd9 | 99426_at, 93221_at |
| AI837110 | Heterogeneous nuclear ribonucleoproteins methyltransferase-like 2 | Hrmt1l2 | 96696_at |
| X04480 | Insulin-like growth factor 1 | Igf1 | 95545_at |
| X71922 | Insulin-like growth factor 2 | Igf2 | 98622_at, 95546_g_at |
| X76291 | Indian hedgehog | Ihh | 103949_at, 98623_g_at |
| J05149 | Insulin receptor | Insr | 102146_at |
| D12645 | Kinesin family member 3A | Kif3a | 100398_at, 161275_at |
| M36775 | Laminin, alpha 1 | Lama1 | 103729_at |
| U12147 | Laminin, alpha 2 | Lama2 | 92366_at |
| X84014 | Laminin, alpha 3 | Lama3 | 97790_s_at, |
| U69176 | Laminin, alpha 4 | Lama4 | 104587_at |
| AV236263 | Laminin, alpha 5 | Lama5 | 161702_f_at |
| AA874589 | LIM and senescent cell antigen-like domains 1 | Lims1 | 104634_at, 161793_at |
| AF064984 | Low density lipoprotein receptor-related protein 5 | Lrp5 | 103806_at, 99931_at |
| AF074265 | Low density lipoprotein receptor-related protein 6 | Lrp6 | 103271_at |
| AV317327 | Mitogen activated protein kinase 1 | Mapk1 | 161583_at |
| Z14249 | Mitogen activated protein kinase 3 | Mapk3 | 101834_at |
| AW120605 | Myeloid/lymphoid or mixed lineage-leukemia translocation to 3 | Mllt3 | 103925_at, 93253_at, 93254_at |
| AA414339 | Nuclear receptor coactivator 6 | Ncoa6 | 95351_at |
| AF074926 | N-deacetylase/N-sulfotransferase (heparan glucosaminyl) 1 | Ndst1 | 92516_at |
| AF091351 | NK2 transcription factor related, locus 5 (Drosophila) | Nkx2-5 | 97777_at, 95525_at |
| U79163 | Noggin | Nog | 97727_at, 93590_at |
| X74134 | Nuclear receptor subfamily 2, group F, member 1 | Nr2f1 | 102715_at |
| X76653 | Nuclear receptor subfamily 2, group F, member 2 | Nr2f2 | 103052_r_at |
| AF010130 | Neuregulin 3 | Nrg3 | 99834_at |
| M20978 | Paired box gene 1 | Pax1 | 96595_at, 161792_f_at |
| X55781 | Paired box gene 2 | Pax2 | 99809_at |
| X59358 | Paired box gene 3 | Pax3 | 100697_at |
| AB010557 | Paired box gene 4 | Pax4 | 99908_at |
| M97013 | Paired box gene 5 | Pax5 | 102578_at |
| X63963 | Paired box gene 6 | Pax6 | 92271_at |
| X57487 | Paired box gene 8 | Pax8 | 96504_at, 96993_at |
| X84000 | Paired box gene 9 | Pax9 | 98838_at |
| M29464 | Platelet derived growth factor, alpha | Pdgfa | 94932_at |
| AI840738 | Platelet derived growth factor receptor, alpha polypeptide | Pdgfra | 160332_at |
| Y15443 | Pleckstrin homology-like domain, family A, member 2 | Phlda2 | 104548_at |
| AI747899 | Phosphatidylinositol transfer protein, beta | Pitpnb | 102696_s_at, 161202_r_at, |
| U70132 | Paired-like homeodomain transcription factor 2 | Pitx2 | 102788_s_at |
| AF027185 | Phospholipase C, gamma 1 | Plcg1 | 98290_at, 102697_at, 104557_at |
| AF000294 | Peroxisome proliferator activated receptor binding protein | Pparbp | 160603_at, 161340_r_at |
| Z67745 | Protein phosphatase 2a, catalytic subunit, alpha isoform | Ppp2ca | 92638_at |
| U77946 | Paired like homeodomain factor 1 | Prop1 | 100698_at |
| AI848841 | Patched homolog 1 | Ptch1 | 104030_at |
| Z22821 | RAB23, member RAS oncogene family | Rab23 | 93718_at |
| AV375524 | V-rel reticuloendotheliosis viral oncogene homolog A (avian) | Rela | 162042_i_at, 104031_at |
| U88566 | Secreted frizzled-related sequence protein 1 | Sfrp1 | 97997_at |
| U88567 | Secreted frizzled-related sequence protein 2 | Sfrp2 | 93503_at, 97813_at |
| AF117709 | Secreted frizzled-related sequence protein 4 | Sfrp4 | 92469_at |
| X76290 | Sonic hedgehog | Shh | 101831_at |
| U66918 | Short stature Homeobox 2 | Shox2 | 99042_s_at |
| AI641895 | Shroom | Shrm | 100024_at |
| U40576 | Single-minded homolog 2 | Sim2 | 92896_s_at |
| U17132 | Solute carrier family 30 (zinc transporter), member 1 | Slc30a1 | 93938_at, 99043_s_at |
| U58992 | MAD homolog 1 (Drosophila) | Smad1 | 102983_at |
| U60530 | MAD homolog 2 (Drosophila) | Smad2 | 104536_at |
| AB008192 | MAD homolog 3 (Drosophila) | Smad3 | 93613_at, 102984_g_at |
| U79748 | MAD homolog 4 (Drosophila) | Smad4 | 160440_at |
| U58993 | MAD homolog 5 (Drosophila) | Smad5 | 102865_at |
| U85614 | SWI/SNF related, matrix associated, actin dependent regulator of | Smarcc1 | 102062_at |
| AF089721 | Smoothened homolog (Drosophila) | Smo | 96812_at |
| AA866668 | SRY-box containing gene 3 | Sox3 | 103301_i_at |
| X51683 | Brachyury | T | 93941_at, 92264_at |
| AF013282 | T-box 15 | Tbx15 | 100354_at, 103302_r_at |
| AA755817 | T-box 2 | Tbx2 | 104655_at, 102256_at |
| AW121328 | T-box 3 | Tbx3 | 103538_at, 100355_g_at |
| U57331 | T-box 6 | Tbx6 | 93611_at, 92705_at |
| AB008174 | Transcription factor 2 | Tcf2 | 101396_at |
| AF035717 | Transcription factor 21 | Tcf21 | 103050_at |
| AI841235 | Transcription factor 3 | Tcf3 | 104458_at, 162159_i_at |
| AJ009862 | Transforming growth factor, beta 1 | Tgfb1 | 101918_at |
| M32745 | Transforming growth factor, beta 3 | Tgfb3 | 102751_at, 160780_at |
| D25540 | Transforming growth factor, beta receptor I | Tgfbr1 | 92427_at |
| X14432 | Thrombomodulin | Thbd | 104601_at, 161382_at |
| AF019048 | Tumor necrosis factor (ligand) superfamily, member 11 | Tnfsf11 | 93416_at |
| AI122079 | Tnf receptor-associated factor 6 | Traf6 | 104189_at, 162023_f_at |
| AB010152 | Transformation related protein 63 | Trp63 | 103810_at, 98874_at |
| L31959 | Tetratricopeptide repeat domain 10 | Ttc10 | 100404_at, 104190_at |
| AW060819 | Twisted gastrulation homolog 1 (Drosophila) | Twsg1 | 102032_at |
| AF089812 | Ubiquitin-conjugating enzyme E2A, RAD6 homolog (S. Cerevisiae) | Ube2a | 96695_at, 162392_r_at |
| AW061016 | Vitamin D receptor | Vdr | 99964_at |
| M95200 | Vascular endothelial growth factor A | Vegfa | 103520_at, 99965_at |
| U73620 | Vascular endothelial growth factor C | Vegfc | 94712_at |
| M11943 | Wingless-related MMTV integration site 1 | Wnt1 | 94134_at |
| U61969 | Wingless related MMTV integration site 10a | Wnt10a | 98862_at |
| AF029307 | Wingless related MMTV integration site 10b | Wnt10b | 92750_s_at, 92752_r_at |
| X70800 | Wingless-related MMTV integration site 11 | Wnt11 | 103490_at, 92751_i_at |
| AF070988 | Wingless related MMTV integration site 2b | Wnt2b | 94126_at |
| M32502 | Wingless-related MMTV integration site 3 | Wnt3 | 99325_at |
| X56842 | Wingless-related MMTV integration site 3A | Wnt3a | 102667_at |
| M89797 | Wingless-related MMTV integration site 4 | Wnt4 | 103238_at |
| M89798 | Wingless-related MMTV integration site 5A | Wnt5a | 99390_at |
| M89799 | Wingless-related MMTV integration site 5B | Wnt5b | 103513_at |
| M89800 | Wingless-related MMTV integration site 6 | Wnt6 | 103735_at |
| M89801 | Wingless-related MMTV integration site 7A | Wnt7a | 101316_at |
| M89802 | Wingless-related MMTV integration site 7B | Wnt7b | 92404_at |
| Z68889 | Wingless-related MMTV integration site 8A | Wnt8a | 99361_at |
| AI553024 | Zinc finger and BTB domain containing 16 | Zbtb16 | 92201_at |
| D70849 | Zinc finger protein of the cerebellum 3 | Zic3 | 98330_at |

### Example 2

### Confirmation of Interdepot Gene Expression Differences by Real Time PCR (RT-PCR)

The differences of expression in genes involved in embryonic development and pattern specification described in Example 1 were confirmed by quantitative RT-PCR.

Analysis of Gene Expression by Real Time PCR

Expression of murine and human genes of particular interest based on the microarray analysis (Tbx15, Shox2, En1, Sfrp2, HoxC9, Nr2f1, Apc4, Thbd, HoxA5 and HoxC8) was further assesses by quantitative real-time RT-PCR. For murine samples, 1 μg of total RNA was reverse transcribed in 20 μl using Advantage RT-for-PCR kit (BD Biosciences, Palo Alto, USA) according manufacturer's instructions. 5 μl of diluted (1/20) reverse transcription reaction was amplified with specific primers (300 nM each) in a 20 μl PCR using a SYBR Green PCR Master Mix (Applied Biosystems, Forest City, USA). For human samples, total RNA was isolated from paired subcutaneous and visceral adipose tissue samples using TRIzol (Life Technologies, Inc., Grand Island, N.Y.), and 1 μg RNA was reverse transcribed with standard reagents (Life Technologies, Inc., Grand Island, N.Y.). 2 μl of each RT reaction was amplified in a 26 μl PCR using the Brilliant SYBR Green QPCR Core Reagent Kit from Stratagene (La Jolla, Calif.). Analysis of murine and human gene expression were assessed in the ABI PRISM 7000 sequence detector for an initial denaturation at 95° C. for 10 minutes, followed by 40 PCR cycles, each cycle consisting of 95° C. for 15 seconds, 60° C. for 1 minute, and 72° C. for 1 minute and SYBR Green fluorescence emissions were monitored after each cycle. For each gene, mRNA expression was calculated relative to 36B4 for human samples and TBP for murine samples. Amplification of specific transcripts was confirmed by melting curve profiles (cooling the sample to 68° C. and heating slowly to 95° C. with measurement of fluorescence) at the end of each PCR. The specificity of the PCR was further verified by subjecting the amplification products to agarose gel electrophoresis. Primer sequences for each gene are given in Table 3.

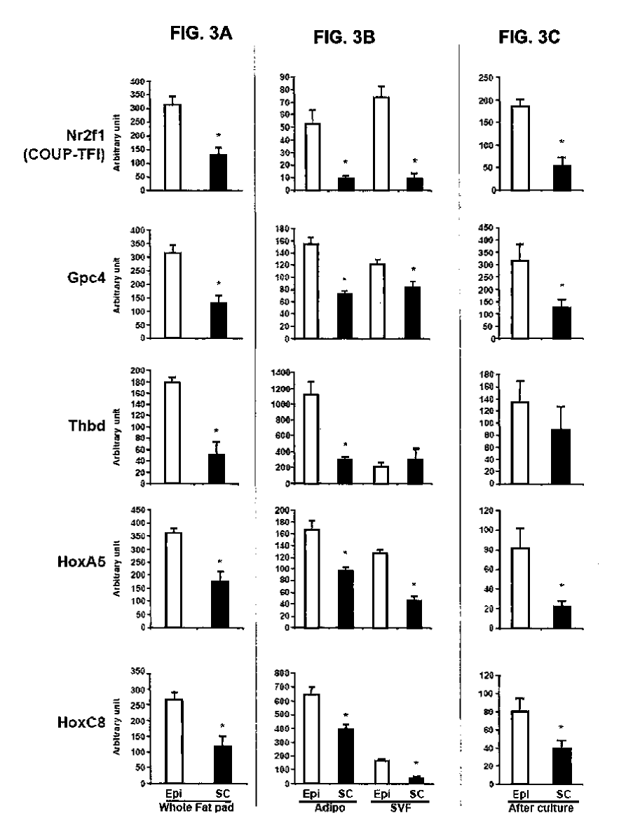

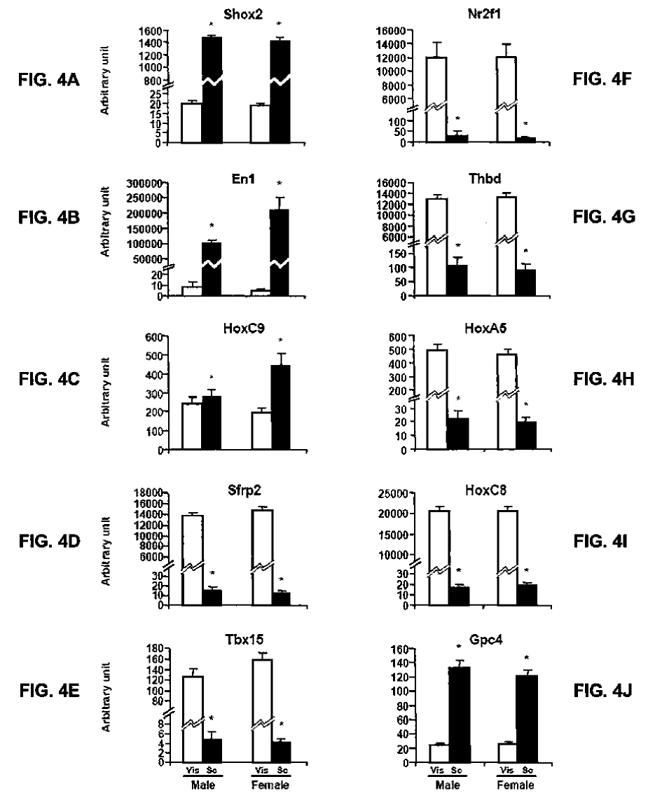

In whole tissue, all predominantly subcutaneous genes Tbx15, Shox2, En1, Sfrp2 and HoxC9 were more highly expressed in subcutaneous adipose tissue as compared to intra-abdominal (epididymal) fat, with the most marked differences observed for Tbx15, Shox2, and En1 expression (39-, 23-, and 5.4-fold respectively; p=0.005, 0.018, and 0.008, respectively) (FIG. 2A). Conversely, all predominant intra-abdominal genes Nr2f1, Gpc4, Thbd, HoxA5 and HoxC8 were significantly more expressed in intra-abdominal adipose tissue as compared to subcutaneous adipose tissue by 2.1- to 3.5-fold (all p<0.05) (FIG. 3A).

Likewise, differences were confirmed in isolated adipocytes and stromovascular cells obtained from both depots by qPCR. Thus, both adipocytes and SVF cells isolated from subcutaneous adipose tissue expressed higher level of all subcutaneous genes Tbx15 [140- and 460-fold (p=0.001 and 0.013)], Shox2 [20- and 205-fold (p=0.006 and 0.012)], En1; [12.3- and 4.9-fold (p=0.0006 and 0.0007)], Sfrp2 [2.6- and 4.5-fold (p=0.001 and 0.04)] and HoxC9 [1.8- and 2.1-fold (p=0.023 and 0.06)] (FIG. 2B). Conversely, adipocytes and SVF from epididymal adipose tissue expressed higher level of intra-abdominal genes Nr2f1, Gpc4, Thbd, HoxA5 and HoxC8 [5.4- and 7.8-fold (p=0.006 and 0.003); 2.1- and 1.5-fold (p=0.003 and 0.05); 3.8- and 0.7-fold (p=0.004 and 0.3); 1.6- and 2.2-fold (p=0.04 and 0.02); 3.8- and 1.7-fold (p=0.009 and 0.02), respectively] (FIG. 3B).

TABLE 3

Primers list for real time PCR

| | | | | | |
|---|---|---|---|---|---|
| | Accession | | SEQ ID | | SEQ ID |
| Name | number | Forward primer (5′-3′) | NO: | Reverse primer (5′-3′) | NO: |

| | | | | | |
|---|---|---|---|---|---|
| T-box 15 | Human: | CGAGTTCATGTGATTCGCAAAG | 1 | TAGGCCGTAACT | 2 |
| (Tbx15) | NM_152380 | | | GTGGTGAACA | |
| | Murine: | TGTTCGCACACTGACCTTTG | 3 | CCAGTGCTGGAG | 4 |
| | NM_009323 | | | GTGGTT | |
| Short stature | Human: | CCGCCAGCCAGTTTGAAG | 5 | GCGCTGTGGCGC | 6 |
| Homeobox 2 | NM_006884 | | | ACGCGC | |
| (Shox2) | Murine: | TGGAACAACTCAA | 7 | TTCAAACTGGCT | 8 |
| | NM_013665 | CGAGCTGGAGA | | AGCGGCTCCTAT | |
| Engrailed 1 | Human: | TTCGGATCGTCCATCCTCC | 9 | GCTCCGTGATGT | 10 |
| (En1) | NM_001426 | | | AGCGGTTT | |
| | Murine: | ACACAACCCTGCGATCCTACTC | 11 | CGCTTGTCTTCCTT | 12 |
| | NM_010133 | | | CTCGTTCT | |
| Secreted | Human: | CCGAAAGGGACCTGAAGAAATC | 13 | GCTCCCCA | 14 |
| frizzled- | NM_003013 | | | CCCTGTTTCTG | |
| related protein | Murine: | AGGACAACGACCTCTGCATC | 15 | TGTCGTCCTC | 16 |
| 2 (Sfrp2) | NM_009144 | | | ATTCTTGGTTT | |
| Homeobox C9 | Human: | CAGCAACCCCGTGGCC | 17 | CCGACGGTCC | 18 |
| (HoxC9) | NM_006897 | | | CTGGTTAAA | |
| | Murine: | CAGCAAGCACAAAGAGGAGA | 19 | CGACGGTCCCTG | 20 |
| | NM_008272 | | | GTTAAATAC | |
| Nuclear | Human: | TCAAAGCCATCGTGCTGTTC | 21 | AGTGCGCACTGG | 22 |
| receptor | NM_005654 | | | AGGAGTACG | |
| subfamily 2, | Murine: | ACATCCGCATCTTTCAGGAAC | 23 | ACAAGCATCTGAC | 24 |
| group F, | NM_010151 | | | GTGAATAGC | |
| member 1 | | | | | |
| (Nr2f1/COUP- | | | | | |
| TFI) | | | | | |
| Glypican 4 | Human: | GCAAGGTCTCCGTGGTAAACC | 25 | CCGGCAGTGGG | 26 |
| (Gpc4) | NM_001448 | | | AGCAGTA | |
| | Murine: | GGCAGCTGGCACTAGTTTG | 27 | AACGGTGCTTGG | 28 |
| | NM_008150 | | | GAGAGAG | |
| Thrombomodulin | Human: | CCCAACACCCAGGCTAGCT | 29 | GATGTCCGTGCA | 30 |
| (Thbd) | NM_000361 | | | GATGAAACC | |
| | Murine: | TCCCAAGTTTCCATGTTTCC | 31 | GCATGAGTTGTG | 32 |
| | NM_009378 | | | TGCTTCGT | |
| Homeobox A5 | Human: | CGCCCAACCCCAGATCTAC | 33 | CGGGCCGCCTATGTTGT | 34 |
| (HoxA5) | NM_019102 | | | | |
| | Murine: | CCCAGATCTACCCCTGGATG | 35 | CAGGGTCTGGT | 36 |
| | NM_010453 | | | AGCGAGTGT | |
| Homeobox C8 | Human: | ATGGATGAGACCCCACGCTC | 37 | AGACTTCAATC | 38 |
| (HoxC8) | NM_022658 | | | CGACGTTTTCG | |
| | Murine: | GTCTCCCAGCCTCATGTTTC | 39 | TCTGATACCGGC | 40 |
| | NM_010466 | | | TGTAAGTTTGT | |
| 36B4 | Human: | AACATGCTCAACATCTCCCC-3 | 41 | CCGACTCCTCC | 42 |
| | NM_001002 | | | GACTCTTC | |
| TATA box- | Murine: | ACCCTTCACCAATGACTCCTATG | 43 | TGACTGCAGCA | 44 |
| binding protein | NM_013684 | | | AATCGCTTGG | |
| (TBP) | | | | | |

### Example 3

### Interdepot Differences in Gene Expression are Independent of Extrinsic Factors

To determine if these differences in gene expression were cell autonomous, preadipocytes (SVF) taken from intra-abdominal (epididymal) or subcutaneous adipose were placed in culture in defined serum free medium and subjected to in vitro differentiation.

Preadipocyte Differentiation

Induction of preadipocyte differentiation was performed using the stromovascular fraction as described by Hauner et al. (Lean, (2000) Proc Nutr Soc 59, 331-6). After 16 hours of incubation, cells were extensively washed with PBS, and the medium was changed into medium consisting on DMEM/F12 1:1 medium with antibiotics supplemented with 33 μM biotin, 17 μM panthotenate, 10 μg/ml human transferrin, 66 nM insulin, 1 nM triiodothyronine, 1 μM dexamethasone, and, for the first 3 days, 1 μg/ml troglitazone. The medium was then changed every 2 days. After 6 days of differentiation, cells were washed once with PBS before proceeding for RNA extraction).

After 6 days, all the predominantly subcutaneous genes and all the predominantly epididymal genes maintained their interdepot differences of expression FIGS. 2C and 3C). Thus, differences of developmental gene expression between depots are independent of extrinsic factors, such as innervation, blood flow, the level of oxygenation and nutrients or any other interstitial factors.

### Example 4

### Interdepot Differences of Expression in Humans

Since the striking interdepot differences for expression of these developmental genes between subcutaneous and intra-abdominal fat in mice appeared to be intrinsic and be present in both the preadipocyte and adipocyte fractions, we decided to determine if similar differences might be present in human adipose tissue. To address this question, 53 lean subjects (22 males and 31 females with BMI <25) with normal fat distribution (WHR for male 0.80-1.06, WHR for female 0.62-0.87) were subjected to abdominal subcutaneous and visceral adipose tissue biopsies and gene expression for the human homologues of each of these developmental genes assessed using real time PCR.

Human Subjects

Paired samples of visceral and subcutaneous adipose tissue were obtained from 198 Caucasian men (n=99) and women (n=99) who underwent open abdominal surgery for gastric banding, cholecystectomy, appendectomy, weight reduction surgery, abdominal injury, or explorative laparotomy. The age ranged from 24 to 85 years for male and from 27 to 86 years for female. Body mass index (BMI) ranged from 21.7 to 46.8 kg/m² for the males and from 20.8 to 54.1 kg/m² for the females. Waist-to-hip ratio (WHR) ranged from 0.8 to 1.37 for the males and from 0.62 to 1.45 for the females. All subjects had a stable weight with no fluctuations of more than 2 percent of the body weight for at least 3 months before surgery. Patients with severe conditions including type 2 diabetes, generalized inflammation or end stage malignant diseases were excluded from the study. Samples of visceral and subcutaneous adipose tissue were immediately frozen in liquid nitrogen after removal. The study was approved by the ethics committee of the University of Leipzig. All subjects gave written informed consent before taking part in the study.

As observed in mice, Nr2f1, Thbd, HoxA5 and HoxC8, which showed higher expression in epididymal fat showed a higher level of expression in visceral adipose tissue of humans, both in males and females (FIGS. 4F, G, H, and I, respectively). In addition, for these genes, the magnitude of interdepot differential gene expression in humans was even greater than that in mice Nr2f1461-fold and 894fold, Thbd 124-fold and 147-fold, HoxA5 23-fold and 24-fold, HoxC8 1210-fold and 1100-fold, for males and females, respectively). Glypican 4 (Gpc4) expression in humans also showed a strong differential expression, however in lean humans this gene was more highly expressed in subcutaneous as compared to visceral adipose tissue with a 5.4-fold difference in males and 4.8-fold difference in females (FIG. 43).

The group of subcutaneous genes also showed significant and differential patterns of expression between depots in humans. In this case, two of the genes, Shox2 and En1, presented a pattern of expression in humans in the same direction as in mice, and in the case of En1, the differential expression was of extreme magnitude (17,500-fold and 42,500-fold for males and females, respectively) (FIGS. 4A-B). As in mice, HoxC9 expression was found significantly higher in subcutaneous than in visceral adipose tissue (2.3-fold), however, in humans this difference was gender-specific and was not present in males (FIG. 4C). Tbx15 and Srfp2 also showed markedly different expression in humans, however in humans these genes were more highly expressed in visceral adipose tissue compared to subcutaneous adipose tissue in both genders (Tbx15: 27.1-fold in male and 38.7-fold in female, Sfrp2: 950-fold in male and 1200-fold in female) (FIGS. 4D-E).

### Example 5

### Gene Expression, BMI and Body Fat Distribution

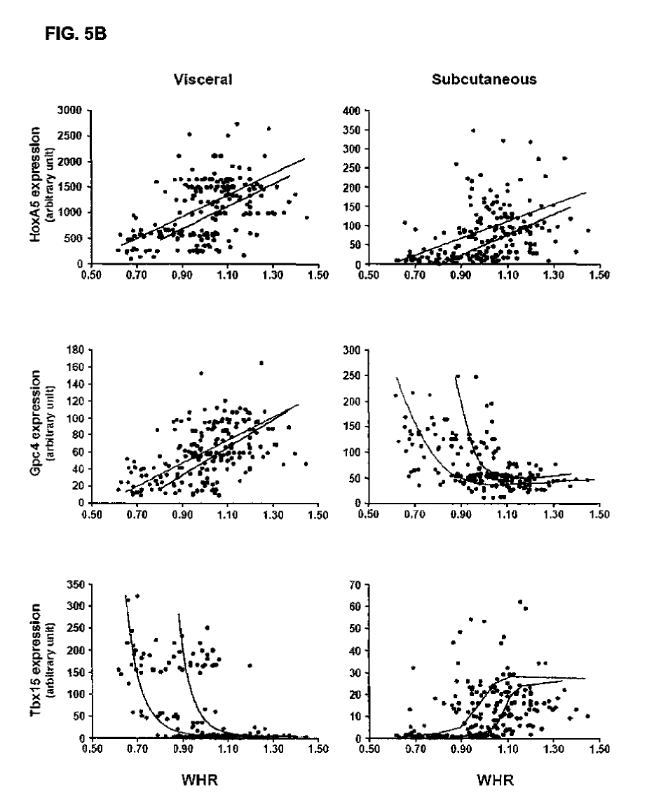

To investigate whether the genes studied were related to obesity or body fat distribution, we determined the level of gene expression in adipose tissue biopsies from this group of 53 subjects plus another group of 145 overweight or obese individuals. The final group of 198 human subjects (99 males and 99 females) ranged from lean to obese (BMI range 21.7-46.8 for male and 20.8-54.1 for female) with variable adipose tissue distribution (Waist-Hip Ratio [WHR] 0.8-1.37 for males and 0.62-1.45 for females) (Table 4). Three of the 10 developmental genes showed significant relationships to BMI or OHR. HoxA5 expression in both visceral and subcutaneous adipose tissue significantly increased with BMI in males (R=0.448, p <0.0001 and, R=0.292, p=0.0034, respectively) and females (R=0.535, p<0.0001 and R=0.361, p=0.0002, respectively) (FIG. 5A). This correlation was more marked in visceral than in subcutaneous adipose tissue in both genders. In addition, there was a significant positive correlation of UoxA5 expression with WER in visceral and subcutaneous adipose tissue for both males (R=0.446, p<0.0001 and R=0.479, p<0.0001, respectively) and females (R=0.580, p<0.0001 and R=0.449, p<0.0001, respectively) (FIG. 5B).

In human adipose, there were very strong correlations of Gpc4 expression with BMI and WHR in both males and females. In this case, the correlation in the two depots was in opposite directions with decreasing Gp4 expression in subcutaneous adipose tissue with increasing BMI (male: R=0.74, p<0.0001; female. R=0.735, p <0.0001) and WHR (male: R=0.575, p<0.0001; female: R=0.730, p<0.0001), and increasing Gpc4 expression in visceral adipose tissue with increasing BMI (male: R=0.525, p<0.0001; female: R=0.507, p<0.0001) and WHR (male: R=0.598, p <0.0001; female: R=0.5, p<0.0001) (FIGS. 5A-B). In addition, the shape of the relationship was different, being fairly linear in visceral adipose tissue, whereas in subcutaneous adipose tissue Gpc4 expression decreased abruptly as individual when from normal BMI (20-25) to overweight (BMI>25) or obese (BMI>30) levels. Likewise, in subcutaneous adipose tissue Gpc4 expression displayed a curvilinear negative correlation with very low levels in males with WHR>1.1 and females with WHR>0.95.

The most profound correlations with BMI and WHR were observed for Tbx15 expression in visceral adipose tissue. As with Gpc4, there was a strong exponential negative relationship with a marked decrease in Tbx15 expression as BMI progressed from normal to overweight or obese levels. This was true in both males (R=0.706, p <0.0001) and females R=0.852, p<0.0001) (FIG. 5A). There was also a strong exponential negative relationship between Tbox15 expression and WHR in visceral adipose tissue with marked declines above WHR of 1.05 for males (R=0.604, p<0.0001) and 0.95 for females (R=0.817, p<0.0001) (FIG. 5B). By contrast, Tbx15 expression showed a more modest positive correlation with both BMI and WHR in subcutaneous adipose tissue of both males (R=0.282, p=0.0047; R=0.406, p<0.0001) and females (R=0.191, p=0.0587; R=0.345, p=0.0005). However, in all cases, expression of Tbx15 in subcutaneous tissue was much lower than the level of expression in visceral adipose tissue of lean individuals. Thus, HoxA5, Gpc4 and Tbx15 expression in adipose tissue were strongly correlated with the level of obesity, as well as adipose tissue distribution, especially Tbx15 expression in visceral fat.

TABLE 4
Characteristics of the Study Population

| Gender | Male (98 subjects) | | Female (98 subjects) | |
|---|---|---|---|---|
| Mean age ± SD (range) years | 56.4 ± 13.3 | (25-85) | 56 ± 16.6 | (27-86) |
| Mean BMI ± SD (range) | 30.8 ± 6.7 | (21.7-46.8) | 31 ± 7.6 | (20.8-54.1) |
| Mean WHR ± SD (range) | 1.07 ± 0.12 | (0.8-1.37) | 0.94 ± 0.19 | (0.62-1.45) |
| Mean fasted Insulin level ± SD (range) pM | 128.8 ± 119.2 | (12-512) | 137.6 ± 129 | (10.5-628) |
| Mean fasted FFA level ± SD (range) mM | 0.53 ± 0.34 | (0.05-1.51) | 0.53 ± 0.32 | (0.05-1.31) |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of diagnosing present or predicting risk of future obesity or undesirable adipose tissue distribution in a subject, the method comprising:
providing a sample comprising a cell from the subject; and
determining a level of mRNA in the cell for one or more genes selected from the genes listed in Table 1,
wherein the level of mRNA indicates whether the subject has, or is at risk of developing, obesity or undesirable adipose tissue distribution.

2. The method of claim 1, wherein the subject is a human.

3. The method of claim 1, wherein the cell is an adipose cell.

4. The method of claim 1, wherein the one or more genes are selected from the group consisting of Tbx15, Shox2, En1, Sfrp2, HoxC9, Nr2f1, Gpc4, Thbd, HoxA5, and HoxC8.

5. The method of claim 1, wherein the one or more genes are selected from the group consisting of HoxA5, Gpc4, and Tbx15.

6. The method of claim 1, further comprising comparing the level to a reference.

7. The method of claim 6, wherein the reference represents a level of mRNA for the selected gene in a subject with a selected BMI.

8. The method of claim 6, wherein the reference represents a level of mRNA for the selected gene in a subject with a BMI above 25.

9. The method of claim 6, wherein the relationship of the levels of the selected gene in the human subject and the reference indicates that the subject has or is likely to develop a BMI above 25.

10. The method of claim 1, wherein the level of the genes is used to select or exclude a subject for participation in a clinical trial.

11. The method of claim 1, further comprising:
giving the subject a treatment or preventive measure for obesity;
providing a second sample comprising a cell from the subject; and
determining a level of mRNA in the second sample for the selected gene or genes,
wherein a difference in the level of mRNA between the first and second samples indicates the subject's response to the treatment or preventive measure for obesity.

12. The method of claim 1, comprising measuring one or both of Tbx15 in visceral fat and Gpc4 in subcutaneous fat.

13. A method of determining a ratio of intra-abdominal (visceral) accumulation of fat versus subcutaneous (peripheral) fat in a subject the method comprising:
providing a first sample from the subject comprising visceral adipose tissue;
providing a second sample from the subject comprising peripheral adipose tissue;
determining a level in the first and second samples of mRNA for one or more genes selected from the genes listed in Table 1;
calculating a ratio of the level of mRNA in the first sample to the level of m RNA in the second sample;
wherein the ratio of the level of mRNA in the first sample to the level of mRNA in the second sample is indicative of the ratio of visceral accumulation of fat versus peripheral fat in the subject.

14. The method of claim 13, wherein the subject is a human.

15. The method of claim 13, wherein the one or more genes are selected from the group consisting of Tbx15, Shox2, En1, Sfrp2, HoxC9, Nr2f1, Gpc4, Thbd, HoxA5, and HoxC8.

16. The method of claim 13, wherein the one or more genes are selected from the group consisting of HoxA5, Gpc4, and Tbx15.

17. A method of identifying a candidate compound for the treatment of obesity, the method comprising:
providing a sample comprising an adipose cell expressing one or more genes selected from the genes listed in Table 1;
contacting the cell with a test compound; and
evaluating the expression of the one or more genes listed in Table 1 in the cell;
wherein a test compound that appropriately modulates die expression of the gene or genes is a candidate compound for the treatment of obesity.

18. The method of claim 17, wherein the adipose cell is from a human.

19. The method of claim 17, wherein the one or more genes are selected from the group consisting of Tbx15, Shox2 En1, Sftp2, HoxC9, Nrf1, Gp04, Thbd, HoxA5, and HoxC8.

20. The method of claim 17, wherein the one or more genes are selected from the group consisting of HoxA5, Gpc4, and Tbx15.

21. A method of identifying a candidate compound for the treatment of obesity, the method comprising:
providing a sample comprising one or more proteins expressed by a gene listed in Table 1;
contacting the sample with a test compound; and
evaluating the activity of the protein in the sample,
wherein a test compound that appropriately modulates the activity of the protein is a candidate compound for the treatment of obesity.

22. The method of claim 21, wherein the subject is a human.

23. The method of claim 21, wherein the one or more genes are selected from the group consisting of Tbx15, Shox2, En1, Sfrp2, HoxC9, Nr2f1, Gpc4, Thbd, HoxA5, and HoxC8.

24. The method of claim 21, wherein the one or more genes are selected from the group consisting of HoxA5, Gpc4, and Tbx15.
